(19) United States
(12) Patent Application Publication
(10) Pub. No.: US 2007/0249633 A1
(43) Pub. Date: Oct. 25, 2007

(54) 1,2,4-Triazolo[1,5a] Pyrimidines and Use Thereof for Controlling Plant-Pathogenic Fungi

(76) Inventors: Carsten Blettner, Mannheim (DE); Markus Gewehr, Kastellaun (DE); Wassilios Grammenos, Ludwigshafen (DE); Thomas Grote, Wachenheim (DE); Udo Hunger, Mainz (DE); Bernd Muller, Frankenthal (DE); Matthias Niedenbruck, Limburgerhof (DE); Joachim Rheinheimer, Ludwigshafen (DE); Peter Schafer, Ottersheim (DE); Frank Schieweck, Hessheim (DE); Anja Schwogler, Mannheim (DE); Oliver Wagner, Neustadt (DE); Michael Rack, Heidelberg (DE); Barbara Nave, Deidesheim (DE); Maria Scherer, Godramstein (DE); Siegfried Strahmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(21) Appl. No.: 11/597,379

(22) PCT Filed: Jun. 8, 2005
(86) PCT No.: PCT/EP05/06171

§ 371 Date: Nov. 22, 2006

(30) Foreign Application Priority Data
Jun. 9, 2004 (DE) 10 2004 028 083.5

(51) Int. Cl. A01N 43/90; C07D 487/04

(52) U.S. Cl. 514/259.1; 544/263

(57) ABSTRACT

The invention relates to triazolopyrimidines of the formula I 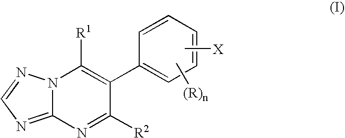
in which the index n and the substituents R, R¹, R² and X are as defined below:
X is nitro, a group —C(S)NR³R⁴, a group —C(═N—OR⁵)(NR⁶R⁷) or a group —C(═N—NR⁸R⁹)(NR¹⁰R¹¹), R is halogen, cyano, hydroxyl, cyanato, alkyl, alkenyl, alkynyl, haloalkyl, haloalkenyl, alkoxy, alkenyloxy, alkynyloxy, haloalkoxy, cycloalkyl, cycloalkenyl, cycloalkoxy, alkoxycarbonyl, alkenyloxycarbonyl, alkynyloxycarbonyl, aminocarbonyl, alkylaminocarbonyl, dialkylaminocarbonyl, alkoximinoalkyl, alkenyloximinocarbonyl, alkynyloximinoalkyl, alkylcarbonyl, alkenylcarbonyl, alkynylcarbonyl, cycloalkylcarbonyl, or a five- to ten-membered saturated, partially unsaturated or aromatic heterocycle which contains one, two, three or four heteroatoms from the group consisting of O, N or S;
R¹ is alkyl in which one carbon atom may be replaced by a silicon atom, alkenyl, alkynyl, cycloalkyl, cycloalkenyl, phenyl, naphthyl, or a five- to ten-membered saturated, partially unsaturated or aromatic heterocycle which is attached via a carbon atom and which contains one, two, three or four heteroatoms, independently of one another selected from the group consisting of O, N and S, as ring members; where R¹ may be substituted as defined in the description;
R² is alkyl, alkenyl or alkynyl which may be substituted as defined in the description;
R³, R⁴, R⁵, R⁶, R⁷, R⁸, R⁹, R¹⁰ and R¹¹ are, independently of one another, selected from the group consisting of hydrogen, alkyl, cycloalkyl, alkenyl or alkynyl, where the 4 lastmentioned radicals may be substituted as defined in the description; or
R³ and R⁴, R⁶ and R⁷, R⁸ and R⁹ and/or R¹⁰ and R¹¹ together with the nitrogen atom to which they are attached form a four-, five- or six-membered saturated or partially unsaturated ring which may be substituted as defined in the description; and n is 0 or an integer 1, 2, 3 or 4; and the agriculturally acceptable salts thereof, to crop protection compositions comprising at least one compound of the formula I and/or an agriculturally acceptable salt thereof and at least one liquid or solid carrier and to a method for controlling phytopathogenic harmful fungi.

The present invention relates to 1,2,4-triazolo[1,5a]pyrimidines, to their use for controlling phytopathogenic fungi and to crop protection compositions comprising, as active component, at least one such compound.

1,2,4-Triazolopyrimidines carrying an optionally substituted phenyl ring in the 6-position, a halogen atom in th 5-position and an amino group in the 7-position have been described in various publications of the prior art, for example in EP-A 71 792, EP-A 550 113, EP-A 834513 and WO-A 98/46608.

WO-A 99/41255 describes similar triazolopyrimidines which, instead of the amino group, carry an optionally substituted aliphatic or cycloaliphatic radical in the 7-position. The compounds are used as fungicides.

WO 03/004465 describes similar fungicidal 1,2,4-triazolo[1,5a]pyrimidines which carry an optionally substituted aliphatic, cycloaliphatic or aromatic radical in the 7-position and, instead of the halogen atom, an optionally substituted alkyl, alkenyl or alkynyl radical in the 5-position.

PCT/EP/03/14283 describes 5-methyl-6-(2-chloro-4-nitrophenyl)-7-(methylbutyl)-1,2,4-triazolo[1,5a]pyrimidine as intermediate for preparing 1,2,4-triazolo-[1,5a]pyrimidine compounds which carry a 4-acylaminophenyl group in the 6-position.

Some of the triazolopyrimidines known from the prior art are, with a view to their fungicidal activity, unsatisfactory, or they have unwanted properties, such as poor compatibility with crop plants.

Accordingly, it is an object of the present invention to provide novel compounds having improved fungicidal activity and/or crop plant compatibility.

Surprisingly, this object is achieved by the triazolopyrimidines of the formula I described below 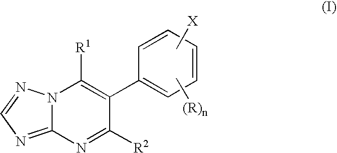
in which the index n and the substituents R, R¹, R² and X are as defined below:
X is nitro, a group —C(S)NR³R⁴, a group —C(═N—OR⁵)(NR⁶R⁷) or a group —C(═N—NR⁸R⁹)(NR¹⁰R¹¹),
R is halogen, cyano, hydroxyl, cyanato, C₁-C₈-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₈-cycloalkenyl, C₃-C₆-cycloalkoxy, C₁-C₈-alkoxycarbonyl, C₂-C₁₀-alkenyloxycarbonyl, C₂-C₁₀-alkynyloxycarbonyl, aminocarbonyl, C₁-C₈-alkylaminocarbonyl, di(C₁-C₈)alkylaminocarbonyl, C₁-C₈-alkoximinoalkyl, C₂-C₁₀-alkenyloximinocarbonyl, C₂-C₁₀-alkynyloximinoalkyl, C₁-C₈-alkylcarbonyl, C₂-C₁₀-alkenylcarbonyl, C₂-C₁₀-alkynylcarbonyl, C₃-C6cycloalkylcarbonyl, or a five- to ten-membered saturated, partially unsaturated or aromatic heterocycle which contains one, two, three or four heteroatoms from the group consisting of O, N and S;
R¹ is C₁-C₁₀-alkyl in which one carbon atom of the alkyl chain may be replaced by a silicon atom, C₂-C₁₀-alkenyl, C₂-C₁₀-alkynyl, C₃-C₁₂-cycloalkyl, C₃-C₁₀-cycloalkenyl, where the two lastmentioned groups may carry a C₁-C₄-alkylidene group, phenyl, naphthyl, or a five- to ten-membered saturated, partially unsaturated or aromatic heterocycle which is attached via a carbon atom and which contains one, two, three or four heteroatoms, independently of one another selected from the group consisting of O, N and S, as ring members;
   where R¹ may be partially or fully halogenated or may carry one, two, three or four groups R^{a}, independently of one another selected from:
   R^{a} halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₁-C₈-alkoximino, C₂-C₁₀-alkenyloximino, C₂-C₁₀-alkynyloximino, aryl-C₁-C₈-alkyloximino, C₂-C₁₀-alkynyl, C₂-C₁₀-alkenyloxycarbonyl, C₂-C₁₀-alkynyloxycarbonyl, phenyl, naphthyl, a five- to ten-membered saturated, partially unsaturated or aromatic heterocycle which contains one, two, three or four heteroatoms, independently of one another selected from the group consisting of O, N and S, as ring members,
   where these aliphatic, alicyclic or aromatic groups for their part may be partially or fully halogenated or may carry one, two or three groups R^{b} independently of one another selected from:
   R^{b} halogen, cyano, nitro, hydroxyl, mercapto, amino, carboxyl, aminocarbonyl, aminothiocarbonyl, alkyl, haloalkyl, alkenyl, alkenyloxy, alkynyl, alkynyloxy, alkoxy, haloalkoxy, alkylthio, alkylamino, dialkylamino, formyl, alkylcarbonyl, alkylsulfinyl, alkylsulfonyl, alkoxycarbonyl, alkylcarbonyloxy, alkylaminocarbonyl, dialkylaminocarbonyl, alkylaminothiocarbonyl and dialkylaminothiocarbonyl, where the alkyl groups in these radicals contain 1 to 6 carbon atoms and the alkenyl or alkynyl groups mentioned in these radicals contain 2 to 8 carbon atoms and the abovementioned groups may be partially or fully halogenated;
   and/or 1, 2 or 3 of the following radicals:
   cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or may carry 1, 2, 3 or 4 alkyl or haloalkyl groups;
R² is C₁-C₄-alkyl, C₂-C₄-alkenyl or C₂-C₄-alkynyl, where the three lastmentioned groups may be unsubstituted or may carry 1, 2, 3, 4, 5 or 6 substituents independently of another selected from the group consisting of halogen, cyano, nitro, C₁-C₂-alkoxy and C₁-C₄-alkoxycarbonyl;
R³, R⁴, R⁵, R⁶, R⁷, R⁸, R⁹, R¹⁰ and R¹¹ are independently of one another selected form the group consisting of hydrogen, C₁-C₆-alkyl, C₃-C₆-cycloalkyl, C₂-C₆-alkenyl or C₂-C₆-alkynyl, where the 4 lastmentioned radicals may carry one, two, three, four, five or six radicals R^{a}; or
R³ and R⁴, R⁶ and R⁷, R⁸ and R⁹, R¹⁰ and R¹¹ together with the nitrogen atom to which are attached form a four-, five- or six-membered saturated or partially unsaturated ring which may carry one, two, three or four substituents independently of one another selected from R^{a};

n is 0 or an integer 1, 2, 3 or 4;

and the agriculturally acceptable salts of the compounds of the formula 1.

Accordingly, the present invention provides the triazolopyrimidine compounds of the formula I and their agriculturally acceptable salts.

The present invention furthermore provides the use of the triazolopyrimidine compounds of the formula I and their agriculturally acceptable salts for controlling phytopathogenic fungi (=harmful fungi), and a method for controlling phytopathogenic fungi wherein the fungi or the materials, plants, the soil or seed to be protected against fungal attack are treated with an effective amount of a triazolopyrimidine compound of the formula I according to the invention and/or with an agriculturally acceptable salt of I.

The present invention furthermore provides a composition for controlling phytopathogenic fungi, which composition comprises at least one compound of the formula I and/or an agriculturally acceptable salt thereof and at least one solid or liquid carrier.

The compounds of the formula I according to the invention differ from the 1,2,4-triazolo[1,5a]pyrimidines known from the publications mentioned above by the substituent X on the phenyl group in the 6-position.

Depending on the substitution pattern, the compounds of the formula I may have one or more centers of chirality, in which case they are present as enantiomer or diastereomer mixtures. The present invention provides both the pure enantiomers or diastereomers and their mixtures. Suitable compounds of the formula I also include all possible stereoisomers (cis/trans isomers) and mixtures thereof.

Agriculturally useful salts are especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting I with an acid of the corresponding anion, preferably of hydrochloric acid, hydrobromic acid, sulfuric acid, phosphoric acid or nitric acid.

In the definitions of the variables given in the formulae above, collective terms are used which are generally representative for the substituents in question. The term Cₙ-Cₘ indicates the number of carbon atoms possible in each case in the substituent or substituent moiety in question:

halogen: fluorine, chlorine, bromine and iodine;

alkyl and all alkyl moieties in alkoxy, alkylamino, alkylaminocarbonyl and alkylaminothiocarbonyl: saturated straight-chain or branched hydrocarbon radicals having 1 to 4, 6, 8 or 10 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl and all haloalkyl moieties in haloalkoxy: straight-chain or branched alkyl groups having 1 to 10 carbon atoms (as mentioned above), where some or all of the hydrogen atoms in these groups may be replaced by halogen atoms as mentioned above, for example C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;

C₁-C₄-alkoxy: an alkyl group having 1 to 4 carbon atoms which is attached via oxygen: for example methoxy, ethoxy, n-propoxy, 1-methylethoxy, butoxy, 1-methylpropoxy, 2-methylpropoxy or 1,1-dimethylethoxy;

C₁-C₆-alkoxy: C₁-C₄-alkoxy as mentioned above, and also, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;

C₁-C₄-haloalkoxy: a C₁-C₄-alkoxy radical as mentioned above which is partially or fully substituted by fluorine, chlorine, bromine and/or iodine, preferably by fluorine, i.e., for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH2—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy;

C₁-C₆-haloalkoxy: C₁-C₄-haloalkoxy as mentioned above, and also, for example, 5-fluoropentoxy, 5-chloropentoxy, 5-bromopentoxy, 5iodopentoxy, undecafluoropentoxy, 6-fluorohexoxy, 6-chlorohexoxy, 6-bromohexoxy, 6-iodohexoxy or tridecafluorohexoxy;

alkylthio: straight-chain or branched alkyl groups having 1 to 10 carbon atoms (as mentioned above) which are attached to the skeleton via a sulfur atom (—S—);

alkylcarbonyl: a straight-chain or branched alkyl group having 1 to 10 carbon atoms (as mentioned above) which is attached to the skeleton via a carbonyl group (—CO—);

alkylsulfinyl: a straight-chain or branched alkyl group having 1 to 10 carbon atoms (as mentioned above) which is attached to the skeleton via a sulfinyl group (—SO—);

alkylsulfonyl: a straight-chain or branched alkyl group having 1 to 10 carbon atoms (as mentioned above) which is attached to the skeleton via a sulfonyl group (—SO₂—);

alkenyl: unsaturated straight-chain or branched hydrocarbon radicals having 2 to 4, 6, 8 or 10 carbon atoms and a double bond in any position, for example C₂-C₆-alkenyl, such as 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl 1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl2-methyl-2-propenyl;

alkenyloxy: alkenyl as mentioned above which is attached via an oxygen atom, for example C₂-C₆alkenyloxy, such as vinyloxy, 1-propenyloxy, 2-propenyloxy, 1-methyl-ethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-i -pentenyloxy, 3-methyl-i -pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl4-pentenyloxy, 2-methyl-4-pentenyloxy, 3-methyl4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy;

haloalkenyl: unsaturated straight-chain or branched hydrocarbon radicals having 2 to 10 carbon atoms and a double bond in any position (as mentioned above), where some or all of the hydrogen atoms in these groups may be replaced by halogen atoms as mentioned above, in particular fluorine, chlorine and bromine;

alkynyl: straight-chain or branched hydrocarbon groups having 2 to 4, 6, 8 or 10 carbon atoms and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

alkynyloxy: alkynyl as mentioned above which is attached via an oxygen atom, for example C₃-C₆-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;

haloalkynyl: unsaturated straight-chain or branched hydrocarbon radicals having 2 to 10 carbon atoms and a triple bond in any position (as mentioned above), where some or all of the hydrogen atoms in these groups may be replaced by halogen atoms as mentioned above, in particular fluorine, chlorine and bromine;

cycloalkyl: mono- or bicyclic saturated hydrocarbon groups having 3 to 6, 8, 10 or 12 carbon ring members, for example C₃-C₈-cycloalkyl, such as cyclopropyl, cyclobutyl, cyclopentyl, cyclohexyl, cycloheptyl and cyclooctyl, or C₇-C₁₂-bicycloalkyl;

cycloalkenyl: mono- or bicyclic monounsaturated hydrocarbon groups having 3 to 10, preferably 8, in particular 6, carbon ring members, such as cyclopenten-1-yl, cyclopenten-3-yl, cyclohexen-1-yl, cyclohexen-3-yl and cyclohexen-4-yl;

aryl: a mono- to tricyclic aromatic ring system which contains 6 to 14 carbon ring members, for example phenyl, naphthyl and anthracenyl;

a five- to ten-membered saturated, partially unsaturated or aromatic heterocycle which is attached via carbon to the triazolopyrimidine and which contains one to four heteroatoms from the group consisting of O, N and S:
5- or 6-membered heterocyclyl, which contains one to three nitrogen atoms and/or one oxygen or sulfur atom or one or two oxygen or sulfur atoms, for example 2-tetrahydrofuranyl, 3-tetrahydrofuranyl, 2-tetrahydrothienyl, 3-tetrahydrothienyl, 2-pyrrolidynyl, 3-pyrrolidynyl, 3-isoxazolidynyl, 4-isoxazolidynyl, 5-isoxazolidynyl, 3-isothiazolidynyl, 4-isothiazolidynyl, 5-isothiazolidynyl, 3-pyrazolidynyl, 4-pyrazolidynyl, 5-pyrazolidynyl, 2-oxazolidynyl, 4-oxazolidynyl, 5-oxazolidynyl, 2-thiazolidynyl, 4-thiazolidynyl, 5-thiazolidynyl, 2-imidazolidynyl, 4-imidazolidynyl, 1,2,4-oxadiazolidin-3-yl, 1,2,4-oxadiazolidin-5-yl, 1,2,4thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidynyl, 3-piperidynyl, 4-piperidynyl, 1,3-dioxan-5-yl, 2-tetrahydropyranyl, 4-tetrahydropyranyl, 2-tetrahydrothienyl, 3-hexahydropyridazynyl, 4-hexahydropyridazynyl, 2-hexahydropyrimidynyl, 4-hexahydropyrimidynyl, 5-hexahydropyrimidynyl, 2-piperazynyl, 1,3,5-hexahydrotriazin-2-yl and 1,2,4-hexahydrotriazin-3-yl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
benzo-fused 5-membered heteroaryl which contains one to three nitrogen atoms or one nitrogen atom and one oxygen or sulfur atom: 5-membered heteroaryl groups which, in addition to carbon atoms, may contain one to four nitrogen atoms or one to three nitrogen atoms and one sulfur or oxygen atom as ring members and in which two ring members may contain and in which two adjacent carbon ring members or one nitrogen and one adjacent carbon ring member may be bridged by a buta-1,3-dien-1,4-diyl group;
6-membered heteroaryl which contains one to three or one to four nitrogen atoms: 6-membered heteroaryl groups which, in addition to carbon atoms, may contain one to three or one to four nitrogen atoms as ring members, for example 2-pyridynyl, 3-pyridynyl, 4-pyridynyl, 3-pyridazynyl, 4-pyridazynyl, 2-pyrimidynyl, 4-pyrimidynyl, 5-pyrimidynyl, 2-pyrazynyl, 1,3,5-triazin-2-yl and 1,2,4-triazin-3-yl;

alkylene: divalent unbranched chains of 1 to 5 CH₂ groups, for example CH₂, CH₂CH₂, CH₂CH₂CH₂, CH₂CH₂CH₂CH₂ and CH₂CH₂CH₂CH₂CH₂;

alkylidene: straight-chain or branched hydrocarbon groups having 1 to 4, preferably 1 or 2, carbon atoms, which groups contain, at one carbon atom, 2 hydrogen atoms less than the parent alkane, for example methylene, ethylidene, propylidene, isopropylidene and butylidene.

With a view to the activity of the compounds of the formula I according to the invention as fungicides, the substituents X, R, R¹, R², R³, R⁴, R⁵, R⁶, R⁷, R⁸, R⁹, R¹⁰, R¹¹, R^{a} and R^{b} and the index n independently of one another and preferably in combination preferably have the meanings given below.

Preference is given to compounds I in which X is nitro or —C(S)NR³R⁴.

In the radical —C(S)NR³R⁴, R³ and R⁴ independently of one another are preferably selected from the group consisting of hydrogen and C₁-C₆-alkyl. R³ and R⁴ independently of one another are in particular selected from the group consisting of hydrogen and C₁-C₄-alkyl such as methyl, ethyl, n-propyl and isopropyl. Particular preference is furthermore given to compounds I in which one of the radicals R³ or R⁴ is hydrogen and the other radical R³ or R⁴ is methyl, ethyl, n-propyl or isopropyl. Particular preference is furthermore given to compounds I in which R³ and R⁴ have the same meaning and are specifically methyl or ethyl. Preference is furthermore given to compounds I in which R³ and R⁴ are each hydrogen.

Especially preferred are compounds I in which X is nitro.

The substituent X may, in principle, be located in any position on the phenyl ring. With a view to the fungicidal activity, preference is given to compounds I in which X is located in the 4-position (para-position) to the point of attachment to the triazolopyrimidine skeleton.

Preference is likewise given to compounds I in which X is located in the 3- or 5-position to the point of attachment to the triazolpyrimidine skeleton.

Preference is given to compounds I in which R is selected from the group consisting of halogen, cyano, C₁-C₆-alkyl, C₁-C₆-haloalkyl, C₁-C₆-alkoxy, Cl-C₈-alkoxycarbonyl and aminocarbonyl.

Particular preference is given to compounds I in which R is selected from the group consisting of fluorine, chlorine, bromine, cyano, C₁-C₄-alkyl, C₁-C₄-haloalkyl, C₁-C₄-alkoxycarbonyl, aminocarbonyl and C₁-C₄-alkoxy.

Especially preferred are compounds I in which R is fluorine, chlorine, C₁-C₂-alkyl, such as methyl or ethyl, C₁-C₂-fluoroalkyl, such as trifluoromethyl, C₁-C₂-alkoxy, such as methoxy, C₁-C₂-alkoxycarbonyl, such as methoxycarbonyl, or aminocarbonyl.

Preference is furthermore given to compounds I in which at least one group R is located in the ortho-position to the point of attachment to the triazolopyrimidine skeleton. Preference is furthermore given to compounds I in which the index n has the value 1, 2 or 3, in particular 1 or 2.

Preference is furthermore given to compounds I in which at least one group R is located in the ortho-position to the point of attachment to the triazolopyrimidine skeleton and the index n has the value 1, 2 or 3, in particular 1 or 2.

(R)n is especially, for example, 2-chloro, 2-fluoro, 2-methyl, 2-methoxy, 2-trifluoromethyl; 2-trifluoromethyl-6-chloro, 2-chloro-6-fluoro, 2,6-difluoro, 2-fluoro-6-methyl, 2,4-difluoro, 2-fluoro-4-chloro, 2-fluoro-3-methyl, 2-fluoro-5-methyl, 2-fluoro-4-methyl, 2-chloro-4-fluoro, 2,4-dichloro, 2-chloro4-methyl, 2-chloro-3methyl, 2-chloro-5-methyl, 2,6-dichloro, 2-chloro-6-methyl, 2-methyl-4-fluoro, 2-methyl-4-chloro, 2,4-dimethyl, 2,3-dimethyl, 2,5-dimethyl or 2,6-dimethyl.

Preference is furthermore given to compounds I in which R¹ is C₁-C₁₀-alkyl in which one carbon atom may be replaced by a silicon atom, C₃-C₈-alkenyl, C₃-C₈-alkynyl, C₃-C₆-cycloalkyl, C₅-C₆-cycloalkenyl, where the two lastmentioned groups may carry a C₁-C₄-alkylidene group, or is a 5- or 6-membered saturated or aromatic heterocycle which is attached via carbon. R¹ may be partially or fully halogenated and may carry one, two, three or four identical or different groups R^{a}.

If R¹ carries one, two, three or four, preferably one, two or three, identical or different groups R^{a}, R^{a} is preferably selected from the group consisting of halogen, cyano, C₁-C₆-alkyl, C₂-C₆-alkenyl, C₂-C₆-alkynyl, C₁-C₆-alkoxy, C₁-C₆-alkoxycarbonyl, C₁-C₆-alkoximino, C₂-C₆-alkenyloximino, C₂-C₆-alkynyloximino, C₃-C₆-cycloalkyl, C₅-C₆-cycloalkenyl, where the aliphatic or alicyclic groups for their part may be partially or fully halogenated or may carry one, two or three groups R^{b}.

If R^{a} carries at least one group R^{b}, R^{b} is preferably selected from the group consisting of halogen, cyano, C₁-C₆-alkyl, C₁-C₆-haloalkyl, C₂-C₆-alkenyl, C₂-C₆-alkynyl, C₁-C₆-alkylcarbonyl, C₁-C₆-haloalkylcarbonyl and C₁-C₆-alkoxy.

Particular preference is given to compounds I in which R¹ is C₁-C₈-alkyl, in particular branched C₃-C₈-alkyl, C₁-C₆-haloalkyl, C₃-C₈-alkenyl, in particular branched C₃-C₈-alkenyl, C₃-C₆-cycloalkyl which may carry a C₁-C₄-alkyl group or C₅-C₆-cycloalkenyl which may carry a C₁-C₄-alkyl group.

Preference is given to compounds I in which R² is C₁-C₄-alkyl which may be substituted by halogen, cyano, nitro or C₁-C₂-alkoxy. Particular preference is given to compounds I in which R² is C₁-C₄-alkyl, especially methyl or ethyl, or C₁-C₄-haloalkyl, especially halomethyl.

Of the remaining radicals, R⁵ is preferably hydrogen or C₁-C₆-alkyl. R⁶ and R⁷ independently of one another are preferably hydrogen or C₁-C₆-alkyl. R⁸, R⁹, R¹⁰ and R¹¹ independently of one another are preferably selected from the group consisting of hydrogen and C₁-C₆-alkyl.

With a view to the selectivity of the compounds I according to the invention, X is preferably nitro. Hereinbelow, such compounds are referred to as compounds I.A.

Preference is likewise given to compounds of the formula I in which X is —C(S)NR³R⁴. Hereinbelow, such compounds are referred to as compounds I.B.

Particular preference is given to triazolopyrimidines of the formulae I.Aa, I.Ab, I.Ba and I.Bb. 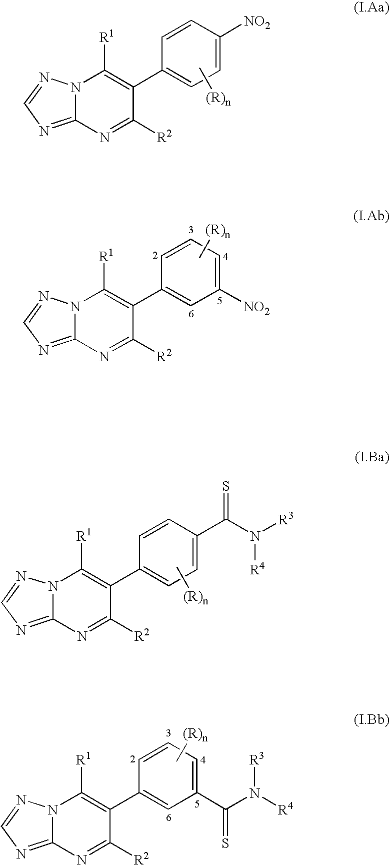
in which the index n and the substituents R, R¹, R², R³ and R⁴ have the meanings mentioned above and in particular the following meanings:
R¹ is C₁-C₁₀-alkyl in which one carbon atom of the alkyl chain may be replaced by a silicon atom, C₃-C₈-alkenyl, C₃-C₈-alkynyl, C₃-C₆-cycloalkyl, C₅-C₆-cycloalkenyl, where the two lastmentioned groups may carry a C₁-C₄-alkylidene group, or is a 5- or 6-membered saturated or aromatic heterocycle which is attached via a carbon; where R¹ may be partially or fully halogenated or may carry one, two, three or four identical or different groups R^{a}, in which
   R^{a} is halogen, cyano, C₁-C₆-alkyl, C₂-C₆-alkenyl, C₂-C₆-alkynyl, C₁-C₆-alkoxy, C₁-C₆-alkoxycarbonyl, C₁-C₆-alkoximino, C₂-C₆-alkenyloximino, C₂-C₆-alkynyloximino, C₃-C₆-cycloalkyl, C₅-C₆-cycloalkenyl in which the aliphatic or alicyclic groups for their part may be partially or fully halogenated or may carry one, two or three groups R^{b};
      R^{b} is halogen, cyano, C₁-C₆-alkyl, C₁-C₆-haloalkyl, C₂-C₆-alkenyl, C₂-C₆-alkynyl, C₁-C₆-alkylcarbonyl, C₁-C₆-haloalkylcarbonyl or C₁-C₆-alkoxy;
R² is C₁-C₄-alkyl which may be substituted by halogen;
R³, R⁴ independently of one another are selected from the group consisting of hydrogen and C₁-C₄-alkyl;
R is halogen, cyano, C₁-C₆-alkyl, C₁-C₆-haloalkyl, C₁-C₆-alkoxy, C₁-C₆-alkoxycarbonyl or aminocarbonyl;
n is 1 or 2.

In particular with a view to their use as fungicides and active compounds for controlling pests, preference is given to the individual compounds compiled in tables 1 to 387 below, which individual compounds are covered by the general formulae I.Aa, I.Ab, I.Ba and I.Bb. The groups mentioned for a substituent in the tables are furthermore per se, independently of the combination in which they are mentioned, a particularly preferred embodiment of the substituent in question.

TABLE A

| No. | R¹ |
|---|---|
| A-1 | CH₃ |
| A-2 | CH₂CH₃ |
| A-3 | CH₂CH₂CH₃ |
| A-4 | CH(CH₃)₂ |
| A-5 | CH₂CH(CH₃)₂ |
| A-6 | (±)CH(CH₃)CH₂CH₃ |
| A-7 | (R)CH(CH₃)CH₂CH₃ |
| A-8 | (S)CH(CH₃)CH₂CH₃ |
| A-9 | (CH₂)₃CH₃ |
| A-10 | C(CH₃)₃ |
| A-11 | (CH₂)₄CH₃ |
| A-12 | CH(CH₂CH₃)₂ |
| A-13 | CH₂CH₂CH(CH₃)₂ |
| A-14 | (±)CH(CH₃)(CH₂)₂CH₃ |
| A-15 | (R)CH(CH₃)(CH₂)₂CH₃ |
| A-16 | (S)CH(CH₃)(CH₂)₂CH₃ |
| A-17 | (±)CH₂CH(CH₃)CH₂CH₃ |
| A-18 | (R)CH₂CH(CH₃)CH₂CH₃ |
| A-19 | (S)CH₂CH(CH₃)CH₂CH₃ |
| A-20 | (±)CH(CH₃)CH(CH₃)₂ |
| A-21 | (R)CH(CH₃)CH(CH₃)₂ |
| A-22 | (S)CH(CH₃)CH(CH₃)₂ |
| A-23 | (CH₂)₅CH₃ |
| A-24 | (±,±)CH(CH₃)CH(CH₃)CH₂CH₃ |
| A-25 | (±,R)CH(CH₃)CH(CH₃)CH₂CH₃ |
| A-26 | (±,S)CH(CH₃)CH(CH₃)CH₂CH₃ |
| A-27 | (R,±)CH(CH₃)CH(CH₃)CH₂CH₃ |
| A-28 | (S,±)CH(CH₃)CH(CH₃)CH₂CH₃ |
| A-29 | (±)CH₂CH(CH₃)CF₃ |
| A-30 | (R)CH₂CH(CH₃)CF₃ |
| A-31 | (S)CH₂CH(CH₃)CF₃ |
| A-32 | (±)CH₂CH(CF₃)CH₂CH₃ |
| A-33 | (R)CH₂CH(CF₃)CH₂CH₃ |
| A-34 | (S)CH₂CH(CF₃)CH₂CH₃ |
| A-35 | (±,±)CH(CH₃)CH(CH₃)CF₃ |
| A-36 | (±,R)CH(CH₃)CH(CH₃)CF₃ |
| A-37 | (±,S)CH(CH₃)CH(CH₃)CF₃ |
| A-38 | (R,±)CH(CH₃)CH(CH₃)CF₃ |
| A-39 | (S,±)CH(CH₃)CH(CH₃)CF₃ |
| A-40 | (±,±)CH(CH₃)CH(CF₃)CH₂CH₃ |
| A-41 | (±,R)CH(CH₃)CH(CF₃)CH₂CH₃ |
| A-42 | (±,S)CH(CH₃)CH(CF₃)CH₂CH₃ |
| A-43 | (R,±)CH(CH₃)CH(CF₃)CH₂CH₃ |
| A-44 | (S,±)CH(CH₃)CH(CF₃)CH₂CH₃ |
| A-45 | CF₃ |
| A-46 | CF₂CF₃ |
| A-47 | CF₂CF₂CF₃ |
| A-48 | c-C₃H₅ |
| A-49 | (1-CH₃)-c-C₃H₄ |
| A-50 | c-C₅H₉ |
| A-51 | c-C₆H₁₁ |
| A-52 | (4-CH₃)-c-C₆H₁₀ |
| A-53 | CH₂C(CH₃)═CH₂ |
| A-54 | CH₂CH₂C(CH₃)═CH₂ |
| A-55 | CH₂—C(CH₃)₃ |
| A-56 | CH₂—Si(CH₃)₃ |
| A-57 | n-C₆H₁₃ |
| A-58 | (CH₂)₃—CH(CH₃)₂ |
| A-59 | (CH₂)₂—CH(CH₃)—C₂H₅ |
| A-60 | CH₂—CH(CH₃)-n-C₃H₇ |
| A-61 | CH(CH₃)-n-C₄H₉ |
| A-62 | CH₂—CH(C₂H₅)₂ |
| A-63 | CH(C₂H₅)-n-C₃H₇ |
| A-64 | CH₂-c-C₅H₉ |
| A-65 | CH₂—CH(CH₃)—CH(CH₃)₂ |
| A-66 | CH(CH₃)—CH₂CH(CH₃)₂ |
| A-67 | CH(CH₃)—CH(CH₃)—C₂H₅ |
| A-68 | CH(CH₃)—C(CH₃)₃ |
| A-69 | (CH₂)₂—C(CH₃)₃ |
| A-70 | CH₂—C(CH₃)₂—C₂H₅ |
| A-71 | 2-CH₃-c-C₅H₈ |
| A-72 | 3-CH₃-c-C₅H₈ |
| A-73 | C(CH₃)₂-n-C₃H₇ |
| A-74 | (CH₂)₆—CH₃ |
| A-75 | (CH₂)₄—CH(CH₃)₂ |
| A-76 | (CH₂)₃—CH(CH₃)—C₂H₅ |
| A-77 | (CH₂)₂—CH(CH₃)-n-C₃H₇ |
| A-78 | CH₂—CH(CH₃)-n-C₄H₉ |
| A-79 | CH(CH₃)-n-C₅H₁₁ |
| A-80 | (CH₂)₃C(CH₃)₃ |
| A-81 | (CH₂)₂CH(CH₃)—CH(CH₃)₂ |
| A-82 | (CH₂)CH(CH₃)—CH₂CH(CH₃)₂ |
| A-83 | CH(CH₃)(CH₂)₂—CH(CH₃)₂ |
| A-84 | (CH₂)₂C(CH₃)₂C₂H₅ |
| A-85 | CH₂CH(CH₃)CH(CH₃)C₂H₅ |
| A-86 | CH(CH₃)CH₂CH(CH₃)C₂H₅ |
| A-87 | CH₂C(CH₃)₂-n-C₃H₇ |
| A-88 | CH(CH₃)CH(CH₃)-n-C₃H₇ |
| A-89 | C(CH₃)₂-n-C₄H₉ |
| A-90 | (CH₂)₂CH(C₂H₅)₂ |
| A-91 | CH₂CH(C₂H₅)-n-C₃H₇ |
| A-92 | CH(C₂H₅)-n-C₄H₉ |
| A-93 | CH₂CH(CH₃)C(CH₃)₃ |
| A-94 | CH(CH₃)CH₂C(CH₃)₃ |
| A-95 | CH₂C(CH₃)₂CH(CH₃)₂ |
| A-96 | CH₂CH(C₂H₅)CH(CH₃)₂ |
| A-97 | CH(CH₃)CH(CH₃)CH(CH₃)₂ |
| A-98 | C(CH₃)₂CH₂CH(CH₃)₂ |
| A-99 | CH(C₂H₅)CH₂CH(CH₃)₂ |
| A-100 | CH(CH₃)C(CH₃)₂C₂H₅ |
| A-101 | CH(CH₃)CH(C₂H₅)₂ |
| A-102 | C(CH₃)₂CH(CH₃)C₂H₅ |
| A-103 | CH(C₂H₅)CH(CH₃)C₂H₅ |
| A-104 | C(CH₃)(C₂H₅)-n-C₃H₇ |
| A-105 | CH(n-C₃H₇)₂ |
| A-106 | CH(n-C₃H₇)CH(CH₃)₂ |
| A-107 | C(CH₃)₂C(CH₃)₃ |
| A-108 | C(CH₃)(C₂H₅)—CH(CH₃)₂ |
| A-109 | C(C₂H₅)₃ |
| A-110 | (3-CH₃)-c-C₆H₁₀ |
| A-111 | (2-CH₃)-c-C₆H₁₀ |
| A-112 | n-C₈H₁₇ |
| A-113 | CH₂C(═NO—CH₃)CH₃ |
| A-114 | CH₂C(═NO—C₂H₅)CH₃ |
| A-115 | CH₂C(═NO-n-C₃H₇)CH₃ |
| A-116 | CH₂C(═NO-i-C₃H₇)CH₃ |
| A-117 | CH(CH₃)C(═NOCH₃)CH₃ |
| A-118 | CH(CH₃)C(═NOC₂H₅)CH₃ |
| A-119 | CH(CH₃)C(═NO-n-C₃H₇)CH₃ |
| A-120 | CH(CH₃)C(═NO-i-C₃H₇)CH₃ |
| A-121 | C(═NOCH₃)C(═NOCH₃)CH₃ |
| A-122 | C(═NOCH₃)C(═NOC₂H₅)CH₃ |
| A-123 | C(═NOCH₃)C(═NO-n-C₃H₇)CH₃ |
| A-124 | C(═NOCH₃)C(═NO-i-C₃H₇)CH₃ |
| A-125 | C(═NOC₂H₅)C(═NOCH₃)CH₃ |
| A-126 | C(═NOC₂H₅)C(═NOC₂H₅)CH₃ |
| A-127 | C(═NOC₂H₅)C(═NO-n-C₃H₇)CH₃ |
| A-128 | C(═NOC₂H₅)C(═NO-i-C₃H₇)CH₃ |
| A-129 | CH₂C(═NO—CH₃)C₂H₅ |
| A-130 | CH₂C(═NO—C₂H₅)C₂H₅ |
| A-131 | CH₂C(═NO-n-C₃H₇)C₂H₅ |
| A-132 | CH₂C(═NO-i-C₃H₇)C₂H₅ |
| A-133 | CH(CH₃)C(═NOCH₃)C₂H₅ |
| A-134 | CH(CH₃)C(═NOC₂H₅)C₂H₅ |
| A-135 | CH(CH₃)C(═NO-n-C₃H₇)C₂H₅ |
| A-136 | CH(CH₃)C(═NO-n-C₃H₇)C₂H₅ |
| A-137 | C(═NOCH₃)C(═NOCH₃)C₂H₅ |
| A-138 | C(═NOCH₃)C(═NOC₂H₅)C₂H₅ |
| A-139 | C(═NOCH₃)C(═NO-n-C₃H₇)C₂H₅ |
| A-140 | C(═NOCH₃)C(═NO-i-C₃H₇)C₂H₅ |
| A-141 | C(═NOC₂H₅)C(═NOCH₃)C₂H₅ |
| A-142 | C(═NOC₂H₅)C(═NOC₂H₅)C₂H₅ |
| A-143 | C(═NOC₂H₅)C(═NO-n-C₃H₇)C₂H₅ |
| A-144 | C(═NOC₂H₅)C(═NO-i-C₃H₇)C₂H₅ |
| A-145 | CH═CH—CH₂CH₃ |
| A-146 | CH₂—CH═CH—CH₃ |
| A-147 | CH₂—CH₂—CH═CH₂ |
| A-148 | C(CH₃)₂CH₂CH₃ |
| A-149 | CH═C(CH₃)₂ |
| A-150 | C(═CH₂)—CH₂CH₃ |
| A-151 | C(CH₃)═CH—CH₃ |
| A-152 | CH(CH₃)CH═CH₂ |
| A-153 | CH═CH-n-C₃H₇ |
| A-154 | CH₂—CH═CH—C₂H₅ |
| A-155 | (CH₂)₂—CH═CH—CH₃ |
| A-156 | (CH₂)₃—CH═CH₂ |
| A-157 | CH═CH—CH(CH₃)₂ |
| A-158 | CH₂—CH═C(CH₃)₂ |
| A-159 | (CH₂)₂—C(CH₃)═CH₂ |
| A-160 | CH═C(CH₃)—C₂H₅ |
| A-161 | CH₂—C(═CH₂)—C₂H₅ |
| A-162 | CH₂—C(CH₃)═CH—CH₃ |
| A-163 | CH₂—CH(CH₃)—CH═CH₂ |
| A-164 | C(═CH₂)—CH₂—CH₂—CH₃ |
| A-165 | C(CH₃)═CH—CH₂—CH₃ |
| A-166 | CH(CH₃)—CH═CH—CH₃ |
| A-167 | CH(CH₃)—CH₂—CH═CH₂ |
| A-168 | C(═CH₂)CH(CH₃)₂ |
| A-169 | C(CH₃)═C(CH₃)₂ |
| A-170 | CH(CH₃)—C(═CH₂)—CH₃ |
| A-171 | C(CH₃)₂—CH═CH₂ |
| A-172 | C(C₂H₅)═CH—CH₃ |
| A-173 | CH(C₂H₅)—CH═CH₂ |
| A-174 | CH═CH—CH₂—CH₂—CH₂—CH₃ |
| A-175 | CH₂—CH═CH—CH₂—CH₂—CH₃ |
| A-176 | CH₂—CH₂—CH═CH—CH₂—CH₃ |
| A-177 | CH₂—CH₂—CH₂—CH═CH—CH₃ |
| A-178 | CH₂—CH₂—CH₂—CH₂—CH═CH₂ |
| A-179 | CH═CH—CH₂—CH(CH₃)CH₃ |
| A-180 | CH₂—CH═CH—CH(CH₃)CH₃ |
| A-181 | CH₂—CH₂—CH═C(CH₃)CH₃ |
| A-182 | CH₂—CH₂—CH₂—C(CH₃)═CH₂ |
| A-183 | CH═CH—CH(CH₃)—CH₂—CH₃ |
| A-184 | CH₂—CH═C(CH₃)—CH₂—CH₃ |
| A-185 | CH₂—CH₂—C(═CH₂)—CH₂—CH₃ |
| A-186 | CH₂—CH₂—C(CH₃)═CH—CH₃ |
| A-187 | CH₂—CH₂—CH(CH₃)—CH═CH₂ |
| A-188 | CH═C(CH₃)—CH₂—CH₂—CH₃ |
| A-189 | CH₂—C(═CH₂)—CH₂—CH₂—CH₃ |
| A-190 | CH₂—C(CH₃)═CH—CH₂—CH₃ |
| A-191 | CH₂—CH(CH₃)—CH═CH—CH₃ |
| A-192 | CH₂—CH(CH₃)—CH₂—CH═CH₂ |
| A-193 | C(═CH₂)—CH₂—CH₂—CH₂—CH₃ |
| A-194 | C(CH₃)═CH—CH₂—CH₂—CH₃ |
| A-195 | CH(CH₃)—CH═CH—CH₂—CH₃ |
| A-196 | CH(CH₃)—CH₂—CH═CH—CH₃ |
| A-197 | CH(CH₃)—CH₂—CH₂—CH═CH₂ |
| A-198 | CH═CH—C(CH₃)₃ |
| A-199 | CH═C(CH₃)—CH(CH₃)—CH₃ |
| A-200 | CH₂—C(═CH₂)—CH(CH₃)—CH₃ |
| A-201 | CH₂—C(CH₃)═C(CH₃)—CH₃ |
| A-202 | CH₂—CH(CH₃)—C(═CH₂)—CH₃ |
| A-203 | C(═CH₂)—CH₂—CH(CH₃)—CH₃ |
| A-204 | C(CH₃)═CH—CH(CH₃)—CH₃ |
| A-205 | CH(CH₃)—CH═C(CH₃)—CH₃ |
| A-206 | CH(CH₃)—CH₂—C(═CH₂)—CH₃ |
| A-207 | CH═C(CH₂—CH₃)—CH₂—CH₃ |
| A-208 | CH₂—C(═CH—CH₃)—CH₂—CH₃ |
| A-209 | CH₂—CH(CH═CH₂)—CH₂—CH₃ |
| A-210 | C(═CH—CH₃)—CH₂—CH₂—CH₃ |
| A-211 | CH(CH═CH₂)—CH₂—CH₂—CH₃ |
| A-212 | C(CH₂—CH₃)═CH—CH₂—CH₃ |
| A-213 | CH(CH₂—CH₃)—CH═CH—CH₃ |
| A-214 | CH(CH₂—CH₃)—CH₂—CH═CH₂ |
| A-215 | CH₂—C(CH₃)₂—CH═CH₂ |
| A-216 | C(═CH₂)—CH(CH₃)—CH₂—CH₃ |
| A-217 | C(CH₃)═C(CH₃)—CH₂—CH₃ |
| A-218 | CH(CH₃)—C(═CH₂)—CH₂—CH₃ |
| A-219 | CH(CH₃)—C(CH₃)═CH—CH₃ |
| A-220 | CH(CH₃)—CH(CH₃)—CH═CH₂ |
| A-221 | C(CH₃)₂—CH═CH—CH₃ |
| A-222 | C(CH₃)₂—CH₂—CH═CH₂ |
| A-223 | C(═CH₂)—C(CH₃)₃ |
| A-224 | C(═CH—CH₃)—CH(CH₃)—CH₃ |
| A-225 | CH(CH═CH₂)—CH(CH₃)—CH₃ |
| A-226 | C(CH₂—CH₃)═C(CH₃)—CH₃ |
| A-227 | CH(CH₂—CH₃)—C(═CH₂)—CH₃ |
| A-228 | C(CH₃)₂—C(═CH₂)—CH₃ |
| A-229 | C(CH₃)(CH═CH₂)—CH₂—CH₃ |
| A-230 | C(CH₃)(CH₂CH₃)—CH₂—CH₂—CH₃ |
| A-231 | CH(CH₂CH₃)—CH(CH₃)—CH₂—CH₃ |
| A-232 | CH(CH₂CH₃)—CH₂—CH(CH₃)—CH₃ |
| A-233 | C(CH₃)₂—C(CH₃)₃ |
| A-234 | C(CH₂—CH₃)—C(CH₃)₃ |
| A-235 | C(CH₃)(CH₂—CH₃)—CH(CH₃)₂ |
| A-236 | CH(CH(CH₃)₂)—CH(CH₃)₂ |
| A-237 | CH═CH—CH₂—CH₂—CH₂—CH₂—CH₃ |
| A-238 | CH₂—CH═CH—CH₂—CH₂—CH₂—CH₃ |
| A-239 | CH₂—CH₂—CH═CH—CH₂—CH₂—CH₃ |
| A-240 | CH₂—CH₂—CH₂—CH═CH—CH₂—CH₃ |
| A-241 | CH₂—CH₂—CH₂—CH₂—CH═CH—CH₃ |
| A-242 | CH₂—CH₂—CH₂—CH₂—CH₂—CH═CH₂ |
| A-243 | CH═CH—CH₂—CH₂—CH(CH₃)—CH₃ |
| A-244 | CH₂—CH═CH—CH₂—CH(CH₃)—CH₃ |
| A-245 | CH₂—CH₂—CH═CH—CH(CH₃)—CH₃ |
| A-246 | CH₂—CH₂—CH₂—CH═C(CH₃)—CH₃ |
| A-247 | CH₂—CH₂—CH₂—CH₂—C(═CH₂)—CH₃ |
| A-248 | CH═CH—CH₂—CH(CH₃)—CH₂—CH₃ |
| A-249 | CH₂—CH═CH—CH(CH₃)—CH₂—CH₃ |
| A-250 | CH₂—CH₂—CH═C(CH₃)—CH₂—CH₃ |
| A-251 | CH₂—CH₂—CH₂—C(═CH₂)—CH₂—CH₃ |
| A-252 | CH₂—CH₂—CH₂—C(CH₃)═CH—CH₃ |
| A-253 | CH₂—CH₂—CH₂—CH(CH₃)—CH═CH₂ |
| A-254 | CH═CH—CH(CH₃)—CH₂—CH₂—CH₃ |
| A-255 | CH₂—CH═C(CH₃)—CH₂—CH₂—CH₃ |
| A-256 | CH₂—CH₂—C(═CH₂)—CH₂—CH₂—CH₃ |
| A-257 | CH₂—CH₂—C(CH₃)═CH—CH₂—CH₃ |
| A-258 | CH₂—CH₂—CH(CH₃)—CH═CH—CH₃ |
| A-259 | CH₂—CH₂—CH(CH₃)—CH₂—CH═CH₂ |
| A-260 | CH═C(CH₃)—CH₂—CH₂—CH₂—CH₃ |
| A-261 | CH₂—C(═CH₂)—CH₂—CH₂—CH₂—CH₃ |
| A-262 | CH₂—C(CH₃)═CH—CH₂—CH₂—CH₃ |
| A-263 | CH₂—CH(CH₃)—CH═CH—CH₂—CH₃ |
| A-264 | CH₂—CH(CH₃)—CH₂—CH═CH—CH₃ |
| A-265 | CH₂—CH(CH₃)—CH₂—CH₂—CH═CH₂ |
| A-266 | C(═CH₂)—CH₂—CH₂—CH₂—CH₂—CH₃ |
| A-267 | C(CH₃)═CH—CH₂—CH₂—CH₂—CH₃ |
| A-268 | CH(CH₃)—CH═CH—CH₂—CH₂—CH₃ |
| A-269 | CH(CH₃)—CH₂—CH═CH—CH₂—CH₃ |
| A-270 | CH(CH₃)—CH₂—CH₂—CH═CH—CH₃ |
| A-271 | CH(CH₃)—CH₂—CH₂—CH₂—CH═CH₂ |
| A-272 | CH═CH—CH₂—C(CH₃)₃ |
| A-273 | CH₂—CH═CH—C(CH₃)₃ |
| A-274 | CH═CH—CH(CH₃)—CH(CH₃)₂ |
| A-275 | CH₂—CH═C(CH₃)—CH(CH₃)₂ |
| A-276 | CH₂—CH₂—C(═CH₂)—CH(CH₃)₂ |
| A-277 | CH₂—CH₂—C(CH₃)═C(CH₃)₂ |
| A-278 | CH₂—CH₂—CH(CH₃)—C(═CH₂)—CH₃ |
| A-279 | CH═C(CH₃)—CH₂—CH(CH₃)₂ |
| A-280 | CH₂—C(═CH₂)—CH₂—CH(CH₃)₂ |
| A-281 | CH₂—C(CH₃)═CH—CH(CH₃)₂ |
| A-282 | CH₂—CH(CH₃)—CH═C(CH₃)₂ |
| A-283 | CH₂—CH(CH₃)—CH₂—C(═CH₂)—CH₃ |
| A-284 | C(═CH₂)—CH₂—CH₂—CH(CH₃)₂ |
| A-285 | C(CH₃)═CH—CH₂—CH(CH₃)₂ |
| A-286 | CH(CH₃)—CH═CH—CH(CH₃)₂ |
| A-287 | CH(CH₃)—CH₂—CH═C(CH₃)₂ |
| A-288 | CH(CH₃)—CH₂—CH₂—C(═CH₂)—CH₃ |
| A-289 | CH═CH—C(CH₃)₂—CH₂—CH₃ |
| A-290 | CH₂—CH₂—C(CH₃)₂—CH═CH₂ |
| A-291 | CH═C(CH₃)—CH(CH₃)—CH₂—CH₃ |
| A-292 | CH₂—C(═CH₂)—CH(CH₃)—CH₂—CH₃ |
| A-293 | CH₂—C(CH₃)═C(CH₃)—CH₂—CH₃ |
| A-294 | CH₂—CH(CH₃)—C(═CH₂)—CH₂—CH₃ |
| A-295 | CH₂—CH(CH₃)—C(CH₃)═CH—CH₃ |
| A-296 | CH₂—CH(CH₃)—CH(CH₃)—CH═CH₂ |
| A-297 | C(═CH₂)—CH₂—CH(CH₃)—CH₂—CH₃ |
| A-298 | C(CH₃)═CH—CH(CH₃)—CH₂—CH₃ |
| A-299 | CH(CH₃)—CH═C(CH₃)—CH₂—CH₃ |
| A-300 | CH(CH₃)—CH₂—C(═CH₂)—CH₂—CH₃ |
| A-301 | CH(CH₃)—CH₂—C(CH₃)═CH—CH₃ |
| A-302 | CH(CH₃)—CH₂—CH(CH₃)—CH═CH₂ |
| A-303 | CH₂—C(CH₃)₂—CH═CH—CH₃ |
| A-304 | CH₂—C(CH₃)₂—CH₂—CH═CH₂ |
| A-305 | C(═CH₂)—CH(CH₃)—CH₂—CH₂—CH₃ |
| A-306 | C(CH₃)═C(CH₃)—CH₂—CH₂—CH₃ |
| A-307 | CH(CH₃)—C(═CH₂)—CH₂—CH₂—CH₃ |
| A-308 | CH(CH₃)—C(CH₃)═CH—CH₂—CH₃ |
| A-309 | CH(CH₃)—CH(CH₃)—CH═CH—CH₃ |
| A-310 | CH(CH₃)—CH(CH₃)—CH₂—CH═CH₂ |
| A-311 | C(CH₃)₂—CH═CH—CH₂—CH₃ |
| A-312 | C(CH₃)₂—CH₂—CH═CH—CH₃ |
| A-313 | C(CH₃)₂—CH₂—CH₂—CH═CH₂ |
| A-314 | CH═CH—CH(CH₂—CH₃)—CH₂—CH₃ |
| A-315 | CH₂—CH═C(CH₂—CH₃)—CH₂—CH₃ |
| A-316 | CH₂—CH₂—C(═CH—CH₃)—CH₂—CH₃ |
| A-317 | CH₂—CH₂—CH(CH═CH₂)—CH₂—CH₃ |
| A-318 | CH═C(CH₂—CH₃)—CH₂—CH₂—CH₃ |
| A-319 | CH₂—C(═CH—CH₃)—CH₂—CH₂—CH₃ |
| A-320 | CH₂—CH(CH═CH₂)—CH₂—CH₂—CH₃ |
| A-321 | CH₂—C(CH₂—CH₃)═CH—CH₂—CH₃ |
| A-322 | CH₂—CH(CH₂—CH₃)—CH═CH—CH₃ |
| A-323 | CH₂—CH(CH₂—CH₃)—CH—CH═CH₂ |
| A-324 | C(═CH—CH₃)—CH₂—CH₂—CH₂—CH₃ |
| A-325 | CH(CH═CH₂)—CH₂—CH₂—CH₂—CH₃ |
| A-326 | C(CH₂—CH₃)═CH—CH₂—CH₂—CH₃ |
| A-327 | CH(CH₂—CH₃)—CH═CH—CH₂—CH₃ |
| A-328 | CH(CH₂—CH₃)—CH₂—CH═CH—CH₃ |
| A-329 | CH(CH₂—CH₃)—CH₂—CH₂—CH═CH₂ |
| A-330 | C(═CH—CH₂—CH₃)—CH₂—CH₂—CH₃ |
| A-331 | C(CH═CH—CH₃)—CH₂—CH₂—CH₃ |
| A-332 | C(CH₂—CH═CH₂)—CH₂—CH₂—CH₃ |
| A-333 | CH═C(CH₃)—C(CH₃)₃ |
| A-334 | CH₂—C(═CH₂)—C(CH₃)₃ |
| A-335 | CH₂—C(CH₃)₂—CH(═CH₂)—CH₃ |
| A-336 | C(═CH₂)—CH(CH₃)—CH(CH₃)—CH₃ |
| A-337 | C(CH₃)═C(CH₃)—CH(CH₃)—CH₃ |
| A-338 | CH(CH₃)—C(═CH₂)—CH(CH₃)—CH₃ |
| A-339 | CH(CH₃)—C(CH₃)═C(CH₃)—CH₃ |
| A-340 | CH(CH₃)—CH(CH₃)—C(═CH₂)—CH₃ |
| A-341 | C(CH₃)₂—CH═C(CH₃)—CH₃ |
| A-342 | C(CH₃)₂—CH₂—C(═CH₂)—CH₃ |
| A-343 | C(CH₃)₂—C(═CH₂)—CH₂—CH₃ |
| A-344 | C(CH₃)₂—C(CH₃)═CH—CH₃ |
| A-345 | C(CH₃)₂—CH(CH₃)CH═CH₂ |
| A-346 | CH(CH₂—CH₃)—CH₂—CH(CH₃)—CH₃ |
| A-347 | CH(CH₂—CH₃)—CH(CH₃)—CH₂—CH₃ |
| A-348 | C(CH₃)(CH₂—CH₃)—CH₂—CH₂—CH₃ |
| A-349 | CH(i-C₃H₇)—CH₂—CH₂—CH₃ |
| A-350 | CH═C(CH₂—CH₃)—CH(CH₃)—CH₃ |
| A-351 | CH₂—C(═CH—CH₃)—CH(CH₃)—CH₃ |
| A-352 | CH₂—CH(CH═CH₂)—CH(CH₃)—CH₃ |
| A-353 | CH₂—C(CH₂—CH₃)═C(CH₃)—CH₃ |
| A-354 | CH₂—CH(CH₂—CH₃)—C(═CH₂)—CH₃ |
| A-355 | CH₂—C(CH₃)(CH═CH₂)—CH₂—CH₃ |
| A-356 | C(═CH₂)—CH(CH₂—CH₃)—CH₂—CH₃ |
| A-357 | C(CH₃)═C(CH₂—CH₃)—CH₂—CH₃ |
| A-358 | CH(CH₃)—C(═CH—CH₃)—CH₂—CH₃ |
| A-359 | CH(CH₃)—CH(CH═CH₂)—CH₂—CH₃ |
| A-360 | CH═C(CH₂—CH₃)—CH(CH₃)—CH₃ |
| A-361 | CH₂—C(═CH—CH₃)—CH(CH₃)—CH₃ |
| A-362 | CH₂—CH(CH═CH₂)—CH(CH₃)—CH₃ |
| A-363 | CH₂—C(CH₂—CH₃)═C(CH₃)—CH₃ |
| A-364 | CH₂—CH(CH₂—CH₃)—C(═CH₂)—CH₃ |
| A-365 | C(═CH—CH₃)—CH₂—CH(CH₃)—CH₃ |
| A-366 | CH(CH═CH₂)—CH₂—CH(CH₃)—CH₃ |
| A-367 | C(CH₂—CH₃)═CH—CH(CH₃)—CH₃ |
| A-368 | CH(CH₂—CH₃)CH═C(CH₃)—CH₃ |
| A-369 | CH(CH₂—CH₃)CH₂—C(═CH₂)—CH₃ |
| A-370 | C(═CH—CH₃)CH(CH₃)—CH₂—CH₃ |
| A-371 | CH(CH═CH₂)CH(CH₃)—CH₂—CH₃ |
| A-372 | C(CH₂—CH₃)═C(CH₃)—CH₂—CH₃ |
| A-373 | CH(CH₂—CH₃)—C(═CH₂)—CH₂—CH₃ |
| A-374 | CH(CH₂—CH₃)—C(CH₃)═CH—CH₃ |
| A-375 | CH(CH₂—CH₃)—CH(CH₃)—CH═CH₂ |
| A-376 | C(CH₃)(CH═CH₂)—CH₂—CH₂—CH₃ |
| A-377 | C(CH₃)(CH₂—CH₃)—CH═CH—CH₃ |
| A-378 | C(CH₃)(CH₂—CH₃)—CH₂—CH═CH₂ |
| A-379 | C[═C(CH₃)—CH₃]—CH₂—CH₂—CH₃ |
| A-380 | CH[C(═CH₂)—CH₃]—CH₂—CH₂—CH₃ |
| A-381 | C(i-C₃H₇)═CH—CH₂—CH₃ |
| A-382 | CH(i-C₃H₇)—CH═CH—CH₃ |
| A-383 | CH(i-C₃H₇)—CH₂—CH═CH₂ |
| A-384 | C(═CH—CH₃)—C(CH₃)₃ |
| A-385 | CH(CH═CH₂)—C(CH₃)₃ |
| A-386 | C(CH₃)(CH═CH₂)CH(CH₃)—CH₃ |
| A-387 | C(CH₃)(CH₂—CH₃)C(═CH₂)—CH₃ |
| A-388 | 2-CH₃-cyclohex-1-enyl |
| A-389 | [2-(═CH₂)]-c-C₆H₉ |
| A-390 | 2-CH₃-cyclohex-2-enyl |
| A-391 | 2-CH₃-cyclohex-3-enyl |
| A-392 | 2-CH₃-cyclohex-4-enyl |
| A-393 | 2-CH₃-cyclohex-5-enyl |
| A-394 | 2-CH₃-cyclohex-6-enyl |
| A-395 | 3-CH₃-cyclohex-1-enyl |
| A-396 | 3-CH₃-cyclohex-2-enyl |
| A-397 | [3-(═CH₂)]-c-C₆H₉ |
| A-398 | 3-CH₃-cyclohex-3-enyl |
| A-399 | 3-CH₃-cyclohex-4-enyl |
| A-400 | 3-CH₃-cyclohex-5-enyl |
| A-401 | 3-CH₃-cyclohex-6-enyl |
| A-402 | 4-CH₃-cyclohex-1-enyl |
| A-403 | 4-CH₃-cyclohex-2-enyl |
| A-404 | 4-CH₃-cyclohex-3-enyl |
| A-405 | [4-(═CH₂)]-c-C₈H₉ |

Table 1

Compounds of the formula I.Aa in which (R)ₙ is 2-chloro, R² is methyl and R¹ for a compound corresponds in each case to one row of table A.

Table 2

Compounds of the formula I.Aa, in which (R)ₙ is 2-fluoro, R² is methyl and R¹ for a compound corresponds in each case to one row of table A.

Table 3

Compounds of the formula I.Aa, in which (R)ₙ is 2-methyl, R² is methyl and R¹ for a compound corresponds in each case to one row of table A.

Table 4

Compounds of the formula I.Aa, in which (R)ₙ is 2,6-dichloro, R² is methyl and R¹ for a compound corresponds in each case to one row of table A.

Table 5

Compounds of the formula I.Aa, in which (R)ₙ is 2,6-difluoro, R² is methyl and R¹ for a compound corresponds in each case to one row of table A.

Table 6

Compounds of the formula I.Aa, in which (R)ₙ is 2,6-dimethyl, R² is methyl and R¹ for a compound corresponds in each case to one row of table A.

Table 7

Compounds of the formula I.Aa, in which (R)ₙ is 2-chloro-3-methyl, R² is methyl and R¹ for a compound corresponds in each case to one row of table A.

Table 8

Compounds of the formula I.Aa, in which (R)ₙ is 2-fluoro-3-methyl, R² is methyl and R¹ for a compound corresponds in each case to one row of table A.

Table 9

Compounds of the formula I.Aa, in which (R)ₙ is 2,3-dimethyl, R² is methyl and R¹ for a compound corresponds in each case to one row of table A.

Table 10

Compounds of the formula I.Aa, in which (R)ₙ is 2-chloro-6-fluoro, R² is methyl and R¹ for a compound corresponds in each case to one row of table A.

Table 11

Compounds of the formula I.Aa, in which (R)ₙ is 2-chloro-6-methyl, R² is methyl and R¹ for a compound corresponds in each case to one row of table A.

Table 12

Compounds of the formula I.Aa, in which (R)ₙ is 2-fluoro-6-methyl, R² is methyl and R¹ for a compound corresponds in each case to one row of table A.

Table 13

Compounds of the formula I.Aa, in which (R)ₙ is 2,5-difluoro, R² is methyl and R¹ for a compound corresponds in each case to one row of table A.

Table 14

Compounds of the formula I.Aa, in which (R)ₙ is 2,3,6-trifluoro, R² is methyl and R¹ for a compound corresponds in each case to one row of table A.

Table 15

Compounds of the formula I.Aa, in which (R)ₙ is 2-chloro-3,6-difluoro, R² is methyl and R¹ for a compound corresponds in each case to one row of table A.

Table 16

Compounds of the formula I.Aa, in which (R)ₙ is 2,5-difluoro-6-methyl, R² is methyl and R¹ for a compound corresponds in each case to one row of table A.

Table 17

Compounds of the formula I.Aa, in which (R)ₙ is 2-fluoro-5-chloro, R² is methyl and R¹ for a compound corresponds in each case to one row of table A.

Table 18

Compounds of the formula I.Aa, in which (R)ₙ is 2,6-difluoro-3-chloro, R² is methyl and R¹ for a compound corresponds in each case to one row of table A.

Table 19

Compounds of the formula I.Aa, in which (R)ₙ is 2,3-dichloro-6-fluoro, R² is methyl and R¹ for a compound corresponds in each case to one row of table A.

Table 20

Compounds of the formula I.Aa, in which (R)ₙ is 2-fluoro-5-chloro-6-methyl, R² is methyl and R¹ for a compound corresponds in each case to one row of table A.

Table 21

Compounds of the formula I.Aa, in which (R)ₙ is 2-fluoro-5-methyl, R² is methyl and R¹ for a compound corresponds in each case to one row of table A.

Table 22

Compounds of the formula I.Aa, in which (R)ₙ is 2,6-difluoro-3-methyl, R² is methyl and R¹ for a compound corresponds in each case to one row of table A.

Table 23

Compounds of the formula I.Aa, in which (R)ₙ is 2-chloro-3-methyl-6-fluoro, R² is methyl and R¹ for a compound corresponds in each case to one row of table A.

Table 24

Compounds of the formula I.Aa, in which (R), is 2-fluoro-5,6dimethyl, R² is methyl and R¹ for a compound corresponds in each case to one row of table A.

Table 25

Compounds of the formula I.Aa, in which (R)ₙ is 2-fluoro-5-methoxy, R² is methyl and R¹ for a compound corresponds in each case to one row of table A.

Table 26

Compounds of the formula I.Aa, in which (R)ₙ is 2,6-difluoro-3-methoxy, R² is methyl and R¹ for a compound corresponds in each case to one row of table A.

Table 27

Compounds of the formula I.Aa, in which (R)ₙ is 2-chloro-3-methoxy-6-fluoro, R² is methyl and R¹ for a compound corresponds in each case to one row of table A.

Table 28

Compounds of the formula I.Aa, in which (R)ₙ is 2-fluoro-5-methoxy-6-methyl, R² is methyl and R¹ for a compound corresponds in each case to one row of table A.

Table 29

Compounds of the formula I.Aa, in which (R)ₙ is 2-fluoro-5-cyano, R² is methyl and R¹ for a compound corresponds in each case to one row of table A.

Table 30

Compounds of the formula I.Aa, in which (R)ₙ is 2,6-difluoro-3-cyano, R² is methyl and R¹ for a compound corresponds in each case to one row of table A.

Table 31

Compounds of the formula I.Aa, in which (R)ₙ is 2-chloro-3-cyano-6fluoro, R² is methyl and R¹ for a compound corresponds in each case to one row of table A.

Table 32

Compounds of the formula I.Aa, in which (R)ₙ is 2-fluoro-5cyanomethyl, R² is methyl and R¹ for a compound corresponds in each case to one row of table A.

Table 33

Compounds of the formula I.Aa, in which (R)ₙ is 2-chloro-5-fluoro, R² is methyl and R¹ for a compound corresponds in each case to one row of table A.

Table 34

Compounds of the formula I.Aa, in which (R)ₙ is 2-chloro-5,6-difluoro, R² is methyl and R¹ for a compound corresponds in each case to one row of table A.

Table 35

Compounds of the formula I.Aa, in which (R)ₙ is 2,6-dichloro-3-fluoro, R² is methyl and R¹ for a compound corresponds in each case to one row of table A.

Table 36

Compounds of the formula I.Aa, in which (R)ₙ is 2-chloro-5-fluoro-6-methyl, R² is methyl and R¹ for a compound corresponds in each case to one row of table A.

Table 37

Compounds of the formula I.Aa, in which (R)ₙ is 2,5-dichloro, R² is methyl and R¹ for a compound corresponds in each case to one row of table A.

Table 38

Compounds of the formula I.Aa, in which (R)ₙ is 2,5-dichloro-6-fluoro, R² is methyl and R¹ for a compound corresponds in each case to one row of table A.

Table 39

Compounds of the formula I.Aa, in which (R)ₙ is 2,3,6-trichloro, R² is methyl and R¹ for a compound corresponds in each case to one row of table A.

Table 40

Compounds of the formula I.Aa, in which (R)ₙ is 2,5-dichloro-6-methyl, R² is methyl and R¹ for a compound corresponds in each case to one row of table A.

Table 41

Compounds of the formula I.Aa, in which (R)ₙ is 2-chloro-5-methyl, R² is methyl and R¹ for a compound corresponds in each case to one row of table A.

Table 42

Compounds of the formula I.Aa, in which (R)ₙ is 2-chloro-5-methyl-6-fluoro, R² is methyl and R¹ for a compound corresponds in each case to one row of table A.

Table 43

Compounds of the formula I.Aa, in which (R)ₙ is 2,6-dichloro-3-methyl, R² is methyl and R¹ for a compound corresponds in each case to one row of table A.

Table 44

Compounds of the formula I.Aa, in which (R)ₙ is 2-chloro-5,6-dimethyl, R² is methyl and R¹ for a compound corresponds in each case to one row of table A.

Table 45

Compounds of the formula I.Aa, in which (R)ₙ is 2-chloro-5-methoxy, R² is methyl and R¹ for a compound corresponds in each case to one row of table A.

Table 46

Compounds of the formula I.Aa, in which (R)ₙ is 2-chloro-5-methoxy-6-fluoro, R² is methyl and R¹ for a compound corresponds in each case to one row of table A.

Table 47

Compounds of the formula I.Aa, in which (R)ₙ is 2,6-dichloro-3-methoxy, R² is methyl and R¹ for a compound corresponds in each case to one row of table A.

Table 48

Compounds of the formula I.Aa, in which (R)ₙ is 2-chloro-5-methoxy-6-methyl, R² is methyl and R¹ for a compound corresponds in each case to one row of table A.

Table 49

Compounds of the formula I.Aa, in which (R)ₙ is 2-chloro-5-cyano, R² is methyl and R¹ for a compound corresponds in each case to one row of table A.

Table 50

Compounds of the formula I.Aa, in which (R)ₙ is 2-chloro-5-cyano-6-fluoro, R² is methyl and R¹ for a compound corresponds in each case to one row of table A.

Table 51

Compounds of the formula I.Aa, in which (R)ₙ is 2,6-dichloro-3-cyano, R² is methyl and R¹ for a compound corresponds in each case to one row of table A.

Table 52

Compounds of the formula I.Aa, in which (R)ₙ is 2-chloro-5-cyano-methyl, R² is methyl and R¹ for a compound corresponds in each case to one row of table A.

Table 53

Compounds of the formula I.Aa, in which (R)ₙ is 2-methyl-5-fluoro, R² is methyl and R¹ for a compound corresponds in each case to one row of table A.

Table 54

Compounds of the formula I.Aa, in which (R)ₙ is 2,3-difluoro-6-methyl, R² is methyl and R¹ for a compound corresponds in each case to one row of table A.

Table 55

Compounds of the formula I.Aa, in which (R)ₙ is 2-chloro-3-fluoro-6-methyl, R² is methyl and R¹ for a compound corresponds in each case to one row of table A.

Table 56

Compounds of the formula I.Aa, in which (R)ₙ is 2,6-dimethyl-3-fluoro, R² is methyl and R¹ for a compound corresponds in each case to one row of table A.

Table 57

Compounds of the formula I.Aa, in which (R)ₙ is 2-methyl-5-chloro, R² is methyl and R¹ for a compound corresponds in each case to one row of table A.

Table 58

Compounds of the formula I.Aa, in which (R)ₙ is 2-fluoro-3-chloro-6-methyl, R² is methyl and R¹ for a compound corresponds in each case to one row of table A.

Table 59

Compounds of the formula I.Aa, in which (R)ₙ is 2,3-dichloro-6-methyl, R² is methyl and R¹ for a compound corresponds in each case to one row of table A.

Table 60

Compounds of the formula I.Aa, in which (R)ₙ is 2,6-dimethyl-3-chloro, R² is methyl and R¹ for a compound corresponds in each case to one row of table A.

Table 61

Compounds of the formula I.Aa, in which (R)ₙ is 2,5-dimethyl, R² is methyl and R¹ for a compound corresponds in each case to one row of table A.

Table 62

Compounds of the formula I.Aa, in which (R)ₙ is 2-fluoro-3,6-dimethyl, R² is methyl and R¹ for a compound corresponds in each case to one row of table A.

Table 63

Compounds of the formula I.Aa, in which (R)ₙ is 2-chloro-3,6-dimethyl, R² is methyl and R¹ for a compound corresponds in each case to one row of table A.

Table 64

Compounds of the formula I.Aa, in which (R)ₙ is 2,3,6-trimethyl, R² is methyl and R¹ for a compound corresponds in each case to one row of table A.

Table 65

Compounds of the formula I.Aa, in which (R)ₙ is 2-methyl-5-methoxy, R² is methyl and R¹ for a compound corresponds in each case to one row of table A.

Table 66

Compounds of the formula I.Aa, in which (R)ₙ is 2-fluoro-3-methoxy-6-methyl, R² is methyl and R¹ for a compound corresponds in each case to one row of table A.

Table 67

Compounds of the formula I.Aa, in which (R)ₙ is 2-chloro-3-methoxy-6-methyl, R² is methyl and R¹ for a compound corresponds in each case to one row of table A.

Table 68

Compounds of the formula I.Aa, in-which (R)ₙ is 2,6-dimethyl-3-methoxy, R² is methyl and R¹ for a compound corresponds in each case to one row of table A.

Table 69

Compounds of the formula I.Aa, in which (R)ₙ is 2-methyl-5-cyano, R² is methyl and R¹ for a compound corresponds in each case to one row of table A.

Table 70

Compounds of the formula I.Aa, in which (R)ₙ is 2-fluoro-3-cyano-6methyl, R² is methyl and R¹ for a compound corresponds in each case to one row of table A.

Table 71

Compounds of the formula I.Aa, in which (R)ₙ is 2-chloro-3-cyano-6-methyl, R² is methyl and R¹ for a compound corresponds in each case to one row of table A.

Table 72

Compounds of the formula I.Aa, in which (R)ₙ is 2,6-dimethyl-3-cyano, R² is methyl and R¹ for a compound corresponds in each case to one row of table A.

Table 73

Compounds of the formula I.Ab, in which (R)ₙ is 6-chloro, R² is methyl and R¹ for a compound corresponds in each case to one row of table A.

Table 74

Compounds of the formula I.Ab, in which (R)ₙ is 6-fluoro, R² is methyl and R¹ for a compound corresponds in each case to one row of table A.

Table 75

Compounds of the formula I.Ab, in which (R)ₙ is 6-methyl, R² is methyl and R¹ for a compound corresponds in each case to one row of table A.

Table 76

Compounds of the formula I.Ab, in which (R)ₙ is 2,6-dichloro, R² is methyl and R¹ for a compound corresponds in each case to one row of table A.

Table 77

Compounds of the formula I.Ab, in which (R)ₙ is 2,6-difluoro, R² is methyl and R¹ for a compound corresponds in each case to one row of table A.

Table 78

Compounds of the formula I.Ab, in which (R)ₙ is 2,6-dimethyl, R² is methyl, and R¹ for a compound corresponds in each case to one row of table A.

Table 79

Compounds of the formula I.Ab, in which (R)ₙ is 3-methyl-6-chloro, R² is methyl and R¹ for a compound corresponds in each case to one row of table A.

Table 80

Compounds of the formula I.Ab, in which (R)ₙ is 3-methyl-6-fluoro, R² is methyl and R¹ for a compound corresponds in each case to one row of table A.

Table 81

Compounds of the formula I.Ab, in which (R)ₙ is 3,6-dimethyl, R² is methyl and R¹ for a compound corresponds in each case to one row of table A.

Table 82

Compounds of the formula I.Ab, in which (R)ₙ is 2-fluoro, R² is methyl and R¹ for a compound corresponds in each case to one row of table A.

Table 83

Compounds of the formula I.Ab, in which (R)ₙ is 2-fluoro6chloro, R² is methyl and R¹ for a compound corresponds in each case to one row of table A.

Table 84

Compounds of the formula I.Ab, in which (R)ₙ is 2-fluoro-6-methyl, R² is methyl and R¹ for a compound corresponds in each case to one row of table A.

Table 85

Compounds of the formula I.Ab, in which (R)ₙ is 2,4-difluoro, R² is methyl and R¹ for a compound corresponds in each case to one row of table A.

Table 86

Compounds of the formula I.Ab, in which (R)ₙ is 2,4,6-trifluoro, R² is methyl and R¹ for a compound corresponds in each case to one row of table A.

Table 87

Compounds of the formula I.Ab, in which (R)ₙ is 2,4difluoro-6-chloro, R² is methyl and R¹ for a compound corresponds in each case to one row of table A.

Table 88

Compounds of the formula I.Ab, in which (R)ₙ is 2,4-difluoro6-methyl, R² is methyl and R¹ for a compound corresponds in each case to one row of table A.

Table 89

Compounds of the formula I.Ab, in which (R), is 2-fluoro-4-chloro, R² is methyl and R¹ for a compound corresponds in each case to one row of table A.

Table 90

Compounds of the formula I.Ab, in which (R)ₙ is 2,6-difluoro-4-chloro, R² is methyl and R¹ for a compound corresponds in each case to one row of table A.

Table 91

Compounds of the formula I.Ab, in which (R)ₙ is 2-fluoro-4,6-dichloro, R² is methyl and R¹ for a compound corresponds in each case to one row of table A.

Table 92

Compounds of the formula I.Ab, in which (R)ₙ is 2-fluoro4-chloro-6-methyl, R² is methyl and R¹ for a compound corresponds in each case to one row of table A.

Table 93

Compounds of the formula I.Ab, in which (R)ₙ is 2-fluoro-4-methyl, R² is methyl and R¹ for a compound corresponds in each case to one row of table A.

Table 94

Compounds of the formula I.Ab, in which (R)ₙ is 2,6-difluoro-4-methyl, R² is methyl and R¹ for a compound corresponds in each case to one row of table A.

Table 95

Compounds of the formula I.Ab, in which (R)ₙ is 2-fluoro-4-methyl-6-chloro, R² is methyl and R¹ for a compound corresponds in each case to one row of table A.

Table 96

Compounds of the formula I.Ab, in which (R)ₙ is 2-fluoro-4,6-dimethyl, R² is methyl and R¹ for a compound corresponds in each case to one row of table A.

Table 97

Compounds of the formula I.Ab, in which (R)ₙ is 2-fluoromethoxy, R² is methyl and R¹ for a compound corresponds in each case to one row of table A.

Table 98

Compounds of the formula I.Ab, in which (R)ₙ is 2,6-difluoro4-methoxy, R² is methyl and R¹ for a compound corresponds in each case to one row of table A.

Table 99

Compounds of the formula I.Ab, in which (R)ₙ is 2-fluoro-4-methoxy-6-chloro, R² is methyl and R¹ for a compound corresponds in each case to one row of table A.

Table 100

Compounds of the formula I.Ab, in which (R)ₙ is 2-fluoro-4-methoxy-6-methyl, R² is methyl and R¹ for a compound corresponds in each case to one row of table A.

Table 101

Compounds of the formula I.Ab, in which (R)ₙ is 2-fluoro-4-cyano, R² is methyl and R¹ for a compound corresponds in each case to one row of table A.

Table 102

Compounds of the formula I.Ab, in which (R)ₙ is 2,6-difluoro4cyano, R² is methyl and R¹ for a compound corresponds in each case to one row of table A.

Table 103

Compounds of the formula I.Ab, in which (R)ₙ is 2-fluoro-4-cyano-6chloro, R² is methyl and R¹ for a compound corresponds in each case to one row of table A.

Table 104

Compounds of the formula I.Ab, in which (R)ₙ is 2-fluoro-4-cyano-6-methyl, R² is methyl and R¹ for a compound corresponds in each case to one row of table A.

Table 105

Compounds of the formula I.Ab, in which (R)ₙ is 2-chloro, R² is methyl and R¹ for a compound corresponds in each case to one row of table A.

Table 106

Compounds of the formula I.Ab, in which (R)ₙ is 2-chloro-6-fluoro, R² is methyl and R¹ for a compound corresponds in each case to one row of table A.

Table 107

Compounds of the formula I.Ab, in which (R)ₙ is 2-chloro-6-methyl, R² is methyl and R¹ for a compound corresponds in each case to one row of table A.

Table 108

Compounds of the formula I.Ab, in which (R)ₙ is 2-chloro4fluoro, R² is methyl and R¹ for a compound corresponds in each case to one row of table A.

Table 109

Compounds of the formula I.Ab, in which (R)ₙ is 2-chloro4,6-difluoro, R² is methyl and R¹ for a compound corresponds in each case to one row of table A.

Table 110

Compounds of the formula I.Ab, in which (R)ₙ is 2,6-dichloro-4-fluoro, R² is methyl and R¹ for a compound corresponds in each case to one row of table A.

Table 111

Compounds of the formula I.Ab, in which (R)ₙ is 2-chloro4-fluoro-6-methyl, R² is methyl and R¹ for a compound corresponds in each case to one row of table A.

Table 112

Compounds of the formula I.Ab, in which (R)ₙ is 2,4-dichloro, R² is methyl and R¹ for a compound corresponds in each case to one row of table A.

Table 113

Compounds of the formula I.Ab, in which (R)ₙ is 2,4-dichloro6fluoro, R² is methyl and R¹ for a compound corresponds in each case to one row of table A.

Table 114

Compounds of the formula I.Ab, in which (R)ₙ is 2,4,6-trichloro, R² is methyl and R¹ for a compound corresponds in each case to one row of table A.

Table 115

Compounds of the formula I.Ab, in which (R)ₙ is 2,4-dichloro-6-methyl, R² is methyl and R¹ for a compound corresponds in each case to one row of table A.

Table 116

Compounds of the formula I.Ab, in which (R)ₙ is 2-chloro-4-methyl, R² is methyl and R¹ for a compound corresponds in each case to one row of table A.

Table 117

Compounds of the formula I.Ab, in which (R)ₙ is 2-chloro-4-methyl-6-fluoro, R² is methyl and R¹ for a compound corresponds in each case to one row of table A.

Table 118

Compounds of the formula I.Ab, in which (R)ₙ is 2,6-dichloro-4-methyl, R² is methyl and R¹ for a compound corresponds in each case to one row of table A.

Table 119

Compounds of the formula I.Ab, in which (R)ₙ is 2-chloro-4,6-dimethyl, R² is methyl and R¹ for a compound corresponds in each case to one row of table A.

Table 120

Compounds of the formula I.Ab, in which (R)ₙ is 2-chloromethoxy, R² is methyl and R¹ for a compound corresponds in each case to one row of table A.

Table 121

Compounds of the formula I.Ab, in which (R)ₙ is 2-chloro-4-methoxy-6-fluoro, R² is methyl and R¹ for a compound corresponds in each case to one row of table A.

Table 122

Compounds of the formula I.Ab, in which (R)ₙ is 2,6-dichloro-4-methoxy, R² is methyl and R¹ for a compound corresponds in each case to one row of table A.

Table 123

Compounds of the formula I.Ab, in which (R)ₙ is 2-chloro-4-methoxy-methyl, R² is methyl and R¹ for a compound corresponds in each case to one row of table A.

Table 124

Compounds of the formula I.Ab, in which (R)ₙ is 2-chloro-4-cyano, R² is methyl and R¹ for a compound corresponds in each case to one row of table A.

Table 125

Compounds of the formula I.Ab, in which (R)ₙ is 2-chloro4cyano-6-fluoro, R² is methyl and R¹ for a compound corresponds in each case to one row of table A.

Table 126

Compounds of the formula I.Ab, in which (R)ₙ is 2,6-dichloro-4-cyano, R² is methyl and R¹ for a compound corresponds in each case to one row of table A.

Table 127

Compounds of the formula I.Ab, in which (R)ₙ is 2-chloro-cyano-&methyl, R² is methyl and R¹ for a compound corresponds in each case to one row of table A.

Table 128

Compounds of the formula I.Ab, in which (R)ₙ is 2-methyl, R² is methyl and R¹ for a compound corresponds in each case to one row of table A.

Table 129

Compounds of the formula I.Ab, in which (R)ₙ is 2-methyl-6-fluoro, R² is methyl and R¹ for a compound corresponds in each case to one row of table A.

Table 130

Compounds of the formula I.Ab, in which (R)ₙ is 2-methyl-6-chloro, R² is methyl and R¹ for a compound corresponds in each case to one row of table A

Table 131

Compounds of the formula I.Ab, in which (R)ₙ is 2-methyl-4-fluoro, R² is methyl and R¹ for a compound corresponds in each case to one row of table A.

Table 132

Compounds of the formula I.Ab, in which (R)ₙ is 2-methyl-4,6-difluoro, R² is methyl and R¹ for a compound corresponds in each case to one row of table A.

Table 133

Compounds of the formula I.Ab, in which (R)ₙ is 2-methyl-4-fluoro-6-chloro, R² is methyl and R¹ for a compound corresponds in each case to one row of table A.

Table 134

Compounds of the formula I.Ab, in which (R)ₙ is 2,6-dimethyl4-fluoro, R² is methyl and R¹ for a compound corresponds in each case to one row of table A.

Table 135

Compounds of the formula I.Ab, in which (R)ₙ is 2-methyl4-chloro, R² is methyl and R¹ for a compound corresponds in each case to one row of table A.

Table 136

Compounds of the formula I.Ab, in which (R)ₙ is 2-methyl4-chloro-6-fluoro, R² is methyl and R¹ for a compound corresponds in each case to one row of table A.

Table 137

Compounds of the formula I.Ab, in which (R)ₙ is 2-methyl4,6-dichloro, R² is methyl and R¹ for a compound corresponds in each case to one row of table A.

Table 138

Compounds of the formula I.Ab, in which (R)ₙ is 2,6-dimethyl4 chloro, R² is methyl and R¹ for a compound corresponds in each case to one row of table A.

Table 139

Compounds of the formula I.Ab, in which (R)ₙ is 2,4-dimethyl, R² is methyl and R¹ for a compound corresponds in each case to one row of table A.

Table 140

Compounds of the formula I.Ab, in which (R)ₙ is 2,4-dimethyl-6-fluoro, R² is methyl and R¹ for a compound corresponds in each case to one row of table A.

Table 141

Compounds of the formula I.Ab, in which (R)ₙ is 2,4-dimethyl-6-chloro, R² is methyl and R¹ for a compound corresponds in each case to one row of table A.

Table 142

Compounds of the formula I.Ab, in which (R)ₙ is 2,4,6-trimethyl, R² is methyl and R¹ for a compound corresponds in each case to one row of table A.

Table 143

Compounds of the formula I.Ab, in which (R)ₙ is 2-methyl-4-methoxy, R² is methyl and R¹ for a compound corresponds in each case to one row of table A.

Table 144

Compounds of the formula I.Ab, in which (R)ₙ is 2-methyl-4-methoxy-6-fluoro, R² is methyl and R¹ for a compound corresponds in each case to one row of table A.

Table 145

Compounds of the formula I.Ab, in which (R)ₙ is 2-methyl-4-methoxy-6-chloro, R² is methyl and R¹ for a compound corresponds in each case to one row of table A.

Table 146

Compounds of the formula I.Ab, in which (R)ₙ is 2,6dimethyl-4-methoxy, R² is methyl and R¹ for a compound corresponds in each case to one row of table A.

Table 147

Compounds of the formula I.Ab, in which (R)ₙ is 2-methyl-4-cyano, R² is methyl and R¹ for a compound corresponds in each case to one row of table A.

Table 148

Compounds of the formula I.Ab, in which (R)ₙ is 2-methyl4-cyano-6-fluoro, R² is methyl and R¹ for a compound corresponds in each case to one row of table A.

Table 149

Compounds of the formula I.Ab, in which (R)ₙ is 2-methyl-4-cyano-6-chloro, R² is methyl and R¹ for a compound corresponds in each case to one row of table A.

Table 150

Compounds of the formula I.Ab, in which (R)ₙ is 2,6-dimethyl-4-cyano, R² is methyl and R¹ for a compound corresponds in each case to one row of table A.

Table 151

Compounds of the formula I.Ba, in which (R)ₙ is 2-chloro, R² is methyl, R³ and R⁴ are each hydrogen and R¹ for a compound corresponds in each case to one row of table A.

Table 152

Compounds of the formula I.Ba, in which (R)ₙ is 2-fluoro, R² is methyl, R³ and R⁴ are each hydrogen and R¹ for a compound corresponds in each case to one row of table A.

Table 153

Compounds of the formula I.Ba, in which (R)ₙ is 2-methyl, R² is methyl, R³ and R⁴ are each hydrogen and R¹ for a compound corresponds in each case to one row of table A.

Table 154

Compounds of the formula I.Ba, in which (R)ₙ is 2,6dichloro, R² is methyl, R³ and R⁴ are each hydrogen and R¹ for a compound corresponds in each case to one row of table A.

Table 155

Compounds of the formula I.Ba, in which (R)ₙ is 2,6-difluoro, R² is methyl, R³ and R⁴ are each hydrogen and R¹ for a compound corresponds in each case to one row of table A.

Table 156

Compounds of the formula I.Ba, in which (R)ₙ is 2,6-dimethyl, R² is methyl, R³ and R⁴ are each hydrogen and R¹ for a compound corresponds in each case to one row of table A.

Table 157

Compounds of the formula I.Ba, in which (R)ₙ is 2chloro-3-methyl, R² is methyl, R³ and R⁴ are each hydrogen and R¹ for a compound corresponds in each case to one row of table A.

Table 158

Compounds of the formula I.Ba, in which (R)ₙ is 2-fluoro-3-methyl, R² is methyl, R³ and R⁴ are each hydrogen and R¹ for a compound corresponds in each case to one row of table A.

Table 159

Compounds of the formula I.Ba, in which (R)ₙ is 2,3-dimethyl, R² is methyl, R³ and R⁴ are each hydrogen and R¹ for a compound corresponds in each case to one row of table A.

Table 160

Compounds of the formula I.Ba, in which (R)ₙ is 2-chloro-6-fluoro, R² is methyl, R³ and R⁴ are each hydrogen and R¹ for a compound corresponds in each case to one row of table A.

Table 161

Compounds of the formula I.Ba, in which (R)ₙ is 2-chloro-6-methyl, R² is methyl, R³ and R⁴ are each hydrogen and R¹ for a compound corresponds in each case to one row of table A.

Table 162

Compounds of the formula I.Ba, in which (R)ₙ is 2-fluoro-6-methyl, R² is methyl, R³ and R⁴ are each hydrogen and R¹ for a compound corresponds in each case to one row of table A.

Table 163

Compounds of the formula I.Ba, in which (R)ₙ is 2,5-difluoro, R² is methyl, R³ and R⁴ are each hydrogen and R¹ for a compound corresponds in each case to one row of table A.

Table 164

Compounds of the formula I.Ba, in which (R)ₙ is 2,3,6-trifluoro, R² is methyl, R³ and R⁴ are each hydrogen and R¹ for a compound corresponds in each case to one row of table A.

Table 165

Compounds of the formula I.Ba, in which (R)ₙ is 2-chloro-3,6-difluoro, R² is methyl, R³ and R⁴ are each hydrogen and R¹ for a compound corresponds in each case to one row of table A.

Table 166

Compounds of the formula I.Ba, in which (R)ₙ is 2,5-difluoro-6-methyl, R² is methyl, R³ and R⁴ are each hydrogen and R¹ for a compound corresponds in each case to one row of table A.

Table 167

Compounds of the formula I.Ba, in which (R)ₙ is 2-fluoro-5-chloro, R² is methyl, R³ and R⁴ are each hydrogen and R¹ for a compound corresponds in each case to one row of table A.

Table 168

Compounds of the formula I.Ba, in which (R)ₙ is 2,6-difluoro-3-chloro, R² is methyl, R³ and R⁴ are each hydrogen and R¹ for a compound corresponds in each case to one row of table A.

Table 169

Compounds of the formula I.Ba, in which (R)ₙ is 2,3-dichloro-6-fluoro, R² is methyl, R³ and R⁴ are each hydrogen and R¹ for a compound corresponds in each case to one row of table A.

Table 170

Compounds of the formula I.Ba, in which (R)ₙ is 2-fluoro-5-chloro-6-methyl, R² is methyl, R³ and R⁴ are each hydrogen and R¹ for a compound corresponds in each case to one row of table A.

Table 171

Compounds of the formula I.Ba, in which (R)ₙ is 2-fluoro-5-methyl, R² is methyl, R³ and R⁴ are each hydrogen and R¹ for a compound corresponds in each case to one row of table A.

Table 172

Compounds of the formula I.Ba, in which (R)ₙ is 2,6-difluoro-3-methyl, R² is methyl, R³ and R⁴ are each hydrogen and R¹ for a compound corresponds in each case to one row of table A.

Table 173

Compounds of the formula I.Ba, in which (R)ₙ is 2-chloro-3-methyl-6-fluoro, R² is methyl, R³ and R⁴ are each hydrogen and R¹ for a compound corresponds in each case to one row of table A.

Table 174

Compounds of the formula I.Ba, in which (R)ₙ is 2-fluoro-5,6-dimethyl, R² is methyl, R³ and R⁴ are each hydrogen and R¹ for a compound corresponds in each case to one row of table A.

Table 175

Compounds of the formula I.Ba, in which (R)ₙ is 2-fluoro-5-methoxy, R² is methyl, R³ and R⁴ are each hydrogen and R¹ for a compound corresponds in each case to one row of table A.

Table 176

Compounds of the formula I.Ba, in which (R)ₙ is 2,6-difluoro-3-methoxy, R² is methyl, R³ and R⁴ are each hydrogen and R¹ for a compound corresponds in each case to one row of table A.

Table 177

Compounds of the formula I.Ba, in which (R)ₙ is 2-chloro3-methoxy-6-fluoro, R² is methyl, R³ and R⁴ are each hydrogen and R¹ for a compound corresponds in each case to one row of table A.

Table 178

Compounds of the formula I.Ba, in which (R)ₙ is 2-fluoro-5-methoxy-6-methyl, R² is methyl, R³ and R⁴ are each hydrogen and R¹ for a compound corresponds in each case to one row of table A.

Table 179

Compounds of the formula I.Ba, in which (R)ₙ is 2-fluoro-5-cyano, R² is methyl, R³ and R⁴ are each hydrogen and R¹ for a compound corresponds in each case to one row of table A.

Table 180

Compounds of the formula I.Ba, in which (R)ₙ is 2,6-difluoro-3-cyano, R² is methyl, R³ and R⁴ are each hydrogen and R¹ for a compound corresponds in each case to one row of table A.

Table 181

Compounds of the formula I.Ba, in which (R)ₙ is 2-chloro-3-cyano-6-fluoro, R² is methyl, R³ and R⁴ are each hydrogen and R¹ for a compound corresponds in each case to one row of table A.

Table 182

Compounds of the formula I.Ba, in which (R)ₙ is 2-fluoro-5-cyano-6-methyl, R² is methyl, R³ and R⁴ are each hydrogen and R¹ for a compound corresponds in each case to one row of table A.

Table 183

Compounds of the formula I.Ba, in which (R)ₙ is 2-chloro-5-fluoro, R² is methyl, R³ and R⁴ are each hydrogen and R¹ for a compound corresponds in each case to one row of table A.

Table 184

Compounds of the formula I.Ba, in which (R)ₙ is 2-chloro-5,6-difluoro, R² is methyl, R³ and R⁴ are each hydrogen and R¹ for a compound corresponds in each case to one row of table A.

Table 185

Compounds of the formula I.Ba, in which (R)ₙ is 2,6-dichloro-3-fluoro, R² is methyl, R³ and R⁴ are each hydrogen and R¹ for a compound corresponds in each case to one row of table A.

Table 186

Compounds of the formula I.Ba, in which (R)ₙ is 2-chloro-5-fluoro-6-methyl, R² is methyl, R³ and R⁴ are each hydrogen and R¹ for a compound corresponds in each case to one row of table A.

Table 187

Compounds of the formula I.Ba, in which (R)ₙ is 2,5-dichloro, R² is methyl, R³ and R⁴ are each hydrogen and R¹ for a compound corresponds in each case to one row of table A.

Table 188

Compounds of the formula I.Ba, in which (R)ₙ is 2,5-dichloro-6-fluoro, R² is methyl, R³ and R⁴ are each hydrogen and R¹ for a compound corresponds in each case to one row of table A.

Table 189

Compounds of the formula I.Ba, in which (R)ₙ is 2,3,6-trichloro, R² is methyl, R³ and R⁴ are each hydrogen and R¹ for a compound corresponds in each case to one row of table A.

Table 190

Compounds of the formula I.Ba, in which (R)ₙ is 2,5-dichloro-6-methyl, R² is methyl, R³ and R⁴ are each hydrogen and R¹ for a compound corresponds in each case to one row of table A.

Table 191

Compounds of the formula I.Ba, in which (R)ₙ is 2-chloro-5-methyl, R² is methyl, R³ and R⁴ are each hydrogen and R¹ for a compound corresponds in each case to one row of table A.

Table 192

Compounds of the formula I.Ba, in which (R)ₙ is 2-chloro-5-methyl-6-fluoro, R² is methyl, R³ and R⁴ are each hydrogen and R¹ for a compound corresponds in each case to one row of table A.

Table 193

Compounds of the formula I.Ba, in which (R)ₙ is 2,6-dichloro-3-methyl, R² is methyl, R³ and R⁴ are each hydrogen and R¹ for a compound corresponds in each case to one row of table A.

Table 194

Compounds of the formula I.Ba, in which (R)ₙ is 2-chloro-5,6-dimethyl, R² is methyl, R³ and R⁴ are each hydrogen and R¹ for a compound corresponds in each case to one row of table A.

Table 195

Compounds of the formula I.Ba, in which (R)ₙ is 2-chloro-5-methoxy, R² is methyl, R³ and R⁴ are each hydrogen and R¹ for a compound corresponds in each case to one row of table A.

Table 196

Compounds of the formula I.Ba, in which (R)ₙ is 2-chloro-5-methoxy-6-fluoro, R² is methyl, R³ and R⁴ are each hydrogen and R¹ for a compound corresponds in each case to one row of table A.

Table 197

Compounds of the formula I.Ba, in which (R)ₙ is 2,6-dichloro-3-methoxy, R² is methyl, R³ and R⁴ are each hydrogen and R¹ for a compound corresponds in each case to one row of table A.

Table 198

Compounds of the formula I.Ba, in which (R)ₙ is 2-chloro-5-methoxy-6-methyl, R² is methyl, R³ and R⁴ are each hydrogen and R¹ for a compound corresponds in each case to one row of table A.

Table 199

Compounds of the formula I.Ba, in which (R)ₙ is 2-chloro-5cyano, R² is methyl, R³ and R⁴ are each hydrogen and R¹ for a compound corresponds in each case to one row of table A.

Table 200

Compounds of the formula I.Ba, in which (R)ₙ is 2-chloro-5-cyano-6-fluoro, R² is methyl, R³ and R⁴ are each hydrogen and R¹ for a compound corresponds in each case to one row of table A.

Table 201

Compounds of the formula I.Ba, in which (R)ₙ is 2,6-dichloro-3-cyano, R² is methyl, R³ and R⁴ are each hydrogen and R¹ for a compound corresponds in each case to one row of table A.

Table 202

Compounds of the formula I.Ba, in which (R)ₙ is 2-chloro-5-cyano-6-methyl, R² is methyl, R³ and R⁴ are each hydrogen and R¹ for a compound corresponds in each case to one row of table A.

Table 203

Compounds of the formula I.Ba, in which (R)ₙ is 2-methyl-5-fluoro, R² is methyl, R³ and R⁴ are each hydrogen and R¹ for a compound corresponds in each case to one row of table A.

Table 204

Compounds of the formula I.Ba, in which (R)ₙ is 2,3-difluoro-&methyl, R² is methyl, R³ and R⁴ are each hydrogen and R¹ for a compound corresponds in each case to one row of table A.

Table 205

Compounds of the formula I.Ba, in which (R)ₙ is 2-chloro-3-fluoro-6-methyl, R² is methyl, R³ and R⁴ are each hydrogen and R¹ for a compound corresponds in each case to one row of table A.

Table 206

Compounds of the formula I.Ba, in which (R)ₙ is 2,6-dimethyl-3-fluoro, R² is methyl, R³ and R⁴ are each hydrogen and R¹ for a compound corresponds in each case to one row of table A.

Table 207

Compounds of the formula I.Ba, in which (R)ₙ is 2-methyl-5-chloro, R² is methyl, R³ and R⁴ are each hydrogen and R¹ for a compound corresponds in each case to one row of table A.

Table 208

Compounds of the formula I.Ba, in which (R)ₙ is 2-fluoro-3-chloro-6-methyl, R² is methyl, R³ and R⁴ are each hydrogen and R¹ for a compound corresponds in each case to one row of table A.

Table 209

Compounds of the formula I.Ba, in which (R)ₙ is 2,3-dichloro-6-methyl, R² is methyl, R³ and R⁴ are each hydrogen and R¹ for a compound corresponds in each case to one row of table A.

Table 210

Compounds of the formula I.Ba, in which (R)ₙ is 2,6-dimethyl-3-chloro, R² is methyl, R³ and R⁴ are each hydrogen and R¹ for a compound corresponds in each case to one row of table A.

Table 211

Compounds of the formula I.Ba, in which (R)ₙ is 2,5-dimethyl, R² is methyl, R³ and R⁴ are each hydrogen and R¹ for a compound corresponds in each case to one row of table A.

Table 212

Compounds of the formula I.Ba, in which (R)ₙ is 2-fluoro-3,6-dimethyl, R² is methyl, R³ and R⁴ are each hydrogen and R¹ for a compound corresponds in each case to one row of table A.

Table 213

Compounds of the formula I.Ba, in which (R)ₙ is 2-chloro-3,6-dimethyl, R² is methyl, R³ and R⁴ are each hydrogen and R¹ for a compound corresponds in each case to one row of table A.

Table 214

Compounds of the formula I.Ba, in which (R)ₙ is 2,3,6-trimethyl, R² is methyl, R³ and R⁴ are each hydrogen and R¹ for a compound corresponds in each case to one row of table A.

Table 215

Compounds of the formula I.Ba, in which (R)ₙ is 2-methyl-5-methoxy, R² is methyl, R³ and R⁴ are each hydrogen and R¹ for a compound corresponds in each case to one row of table A.

Table 216

Compounds of the formula I.Ba, in which (R)ₙ is 2-fluoro-3-methoxy-6-methyl, R² is methyl, R³ and R⁴ are each hydrogen and R¹ for a compound corresponds in each case to one row of table A.

Table 217

Compounds of the formula I.Ba, in which (R)ₙ is 2-chloro-3-methoxy-6-methyl, R² is methyl, R³ and R⁴ are each hydrogen and R¹ for a compound corresponds in each case to one row of table A.

Table 218

Compounds of the formula I.Ba, in which (R)ₙ is 2,6-dimethyl-3-methoxy, R² is methyl, R³ and R⁴ are each hydrogen and R¹ for a compound corresponds in each case to one row of table A.

Table 219

Compounds of the formula I.Ba, in which (R)ₙ is 2-methyl-5-cyano, R² is methyl, R³ and R⁴ are each hydrogen and R¹ for a compound corresponds in each case to one row of table A.

Table 220

Compounds of the formula I.Ba, in which (R)ₙ is 2-fluoro-3-cyano-6-methyl, R² is methyl, R³ and R⁴ are each hydrogen and R¹ for a compound corresponds in each case to one row of table A.

Table 221

Compounds of the formula I.Ba, in which (R)ₙ is 2-chloro-3-cyano-6-methyl, R² is methyl, R³ and R⁴ are each hydrogen and R¹ for a compound corresponds in each case to one row of table A.

Table 222

Compounds of the formula I.Ba, in which (R)ₙ is 2,6-dimethyl-3-cyano, R² is methyl, R³ and R⁴ are each hydrogen and R¹ for a compound corresponds in each case to one row of table A.

Table 223

Compounds of the formula I.Ba, in which (R)ₙ is 2-chloro, R² is methyl, R³ and R⁴ are each methyl and R¹ for a compound corresponds in each case to one row of table A.

Table 224

Compounds of the formula I.Ba, in which (R)ₙ is 2-fluoro, R² is methyl, R³ and R⁴ are each methyl and R¹ for a compound corresponds in each case to one row of table A.

Table 225

Compounds of the formula I.Ba, in which (R)ₙ is 2-methyl, R² is methyl, R³ and R⁴ are each methyl and R¹ for a compound corresponds in each case to one row of table A.

Table 226

Compounds of the formula I.Ba, in which (R)ₙ is 2,6-dichloro, R² is methyl, R³ and R⁴ are each methyl and R¹ for a compound corresponds in each case to one row of table A.

Table 227

Compounds of the formula I.Ba, in which (R)ₙ is 2,6-difluoro, R² is methyl, R³ and R⁴ are each methyl and R¹ for a compound corresponds in each case to one row of table A.

Table 228

Compounds of the formula I.Ba, in which (R)ₙ is 2,6dimethyl, R² is methyl, R³ and R⁴ are each methyl and R¹ for a compound corresponds in each case to one row of table A.

Table 229

Compounds of the formula I.Ba, in which (R)ₙ is 2-chloro-3-methyl, R² is methyl, R³ and R⁴ are each methyl and R¹ for a compound corresponds in each case to one row of table A.

Table 230

Compounds of the formula I.Ba, in which (R)ₙ is 2-fluoro-3-methyl, R² is methyl, R³ and R⁴ are each methyl and R¹ for a compound corresponds in each case to one row of table A.

Table 231

Compounds of the formula I.Ba, in which (R)ₙ is 2,3-dimethyl, R² is methyl, R³ and R⁴ are each methyl and R¹ for a compound corresponds in each case to one row of table A.

Table 232

Compounds of the formula I.Ba, in which (R)ₙ is 2-chloro-6-fluoro, R² is methyl, R³ and R⁴ are each methyl and R¹ for a compound corresponds in each case to one row of table A.

Table 233

Compounds of the formula I.Ba, in which (R)ₙ is 2-chloro-methyl, R² is methyl, R³ and R⁴ are each methyl and R¹ for a compound corresponds in each case to one row of table A.

Table 234

Compounds of the formula I.Ba, in which (R)ₙ is 2-fluoro-6-methyl, R² is methyl, R³ and R⁴ are each methyl and R¹ for a compound corresponds in each case to one row of table A.

Table 235

Compounds of the formula I.Ba, in which (R)ₙ is 2-chloro, R² is methyl, R³ is hydrogen, R⁴is methyl and R¹ for a compound corresponds in each case to one row of table A.

Table 236

Compounds of the formula I.Ba, in which (R)ₙ is 2-fluoro, R² is methyl, R³ is hydrogen, R⁴ is methyl and R¹ for a compound corresponds in each case to one row of table A.

Table 237

Compounds of the formula I.Ba, in which (R)ₙ is 2-methyl, R² is methyl, R³ is hydrogen, R⁴ is methyl and R¹ for a compound corresponds in each case to one row of table A.

Table 238

Compounds of the formula I.Ba, in which (R)ₙ is 2,6-dichloro, R² is methyl, R³ is hydrogen, R⁴ is methyl and R¹ for a compound corresponds in each case to one row of table A.

Table 239

Compounds of the formula I.Ba, in which (R)ₙ is 2,6-difluoro, R² is methyl, R³ is hydrogen, R⁴ is methyl and R¹ for a compound corresponds in each case to one row of table A.

Table 240

Compounds of the formula I.Ba, in which (R)ₙ is 2,6-dimethyl, R² is methyl, R³ is hydrogen, R⁴is methyl and R¹ for a compound corresponds in each case to one row of table A.

Table 241

Compounds of the formula I.Ba, in which (R)ₙ is 2-chloro-3-methyl, R² is methyl, R³ is hydrogen, R⁴is methyl and R¹ for a compound corresponds in each case to one row of table A.

Table 242

Compounds of the formula I.Ba, in which (R)ₙ is 2-fluoro-3-methyl, R² is methyl, R³ is hydrogen, R⁴ is methyl and R¹ for a compound corresponds in each case to one row of table A.

Table 243

Compounds of the formula I.Ba, in which (R)ₙ is 2,3-dimethyl, R² is methyl, R³ is hydrogen, R⁴ is methyl and R¹ for a compound corresponds in each case to one row of table A.

Table 244

Compounds of the formula I.Ba, in which (R)ₙ is 2-chloro-6-fluoro, R² is methyl, R³ is hydrogen, R⁴ is methyl and R¹ for a compound corresponds in each case to one row of table A.

Table 245

Compounds of the formula I.Ba, in which (R)ₙ is 2-chloro-6-methyl, R² is methyl, R³ isn hydrogen, R⁴ is methyl and R¹ for a compound corresponds in each case to one row of table A.

Table 246

Compounds of the formula I.Ba, in which (R)ₙ is 2-fluoro-6-methyl, R² is methyl, R³ is hydrogen, R⁴ is methyl and R¹ for a compound corresponds in each case to one row of table A.

Table 247

Compounds of the formula I.Ba, in which (R)ₙ is 2-chloro, R² is methyl, R³ is hydrogen, R⁴ is isopropyl and R¹ for a compound corresponds in each case to one row of table A.

Table 248

Compounds of the formula I.Ba, in which (R)ₙ is 2-fluoro, R² is methyl, R³ is hydrogen, R⁴ is isopropyl and R¹ for a compound corresponds in each case to one row of table A.

Table 249

Compounds of the formula I.Ba, in which (R)ₙ is 2-methyl, R² is methyl, R³ is hydrogen, R⁴ is isopropyl and R¹ for a compound corresponds in each case to one row of table A.

Table 250

Compounds of the formula I.Ba, in which (R)ₙ is 2,6-dichloro, R² is methyl, R³ is hydrogen, R⁴ is isopropyl and R¹ for a compound corresponds in each case to one row of table A.

Table 251

Compounds of the formula I.Ba, in which (R)ₙ is 2,6-difluoro, R² is methyl, R³ is hydrogen, R⁴ is isopropyl and R¹ for a compound corresponds in each case to one row of table A.

Table 252

Compounds of the formula I.Ba, in which (R)ₙ is 2,6dimethyl, R² is methyl, R³ is hydrogen, R⁴ is isopropyl and R¹ for a compound corresponds in each case to one row of table A.

Table 253

Compounds of the formula I.Ba, in which (R)ₙ is 2-chloro-3-methyl, R² is methyl, R³ is hydrogen, R⁴ is isopropyl and R¹ for a compound corresponds in each case to one row of table A.

Table 254

Compounds of the formula I.Ba, in which (R)ₙ is 2-fluoro-3-methyl, R² is methyl, R³ is hydrogen, R⁴ is isopropyl and R¹ for a compound corresponds in each case to one row of table A.

Table 255

Compounds of the formula I.Ba, in which (R)ₙ is 2,3-dimethyl, R² is methyl, R³ is hydrogen, R¹ is isopropyl and R¹ for a compound corresponds in each case to one row of table A.

Table 256

Compounds of the formula I.Ba, in which (R)ₙ is 2-chloro-6-fluoro, R² is methyl, R³ is hydrogen, R⁴ is isopropyl and R¹ for a compound corresponds in each case to one row of table A.

Table 257

Compounds of the formula I.Ba, in which (R)ₙ is 2-chloro-6methyl, R² is methyl, R³ is hydrogen, R⁴ is isopropyl and R¹ for a compound corresponds in each case to one row of table A.

Table 258

Compounds of the formula I.Ba, in which (R)ₙ is 2-fluoro-6-methyl, R² is methyl, R³ is hydrogen, R⁴ is isopropyl and R¹ for a compound corresponds in each case to one row of table A.

Table 259

Compounds of the formula I.Bb, in which (R)ₙ is 6-chloro, R² is methyl, R³ and R⁴ are each hydrogen and R¹ for a compound corresponds in each case to one row of table A.

Table 260

Compounds of the formula I.Bb, in which (R)ₙ is 6-fluoro, R² is methyl, R³ and R⁴ are each hydrogen and R¹ for a compound corresponds in each case to one row of table A.

Table 261

Compounds of the formula I.Bb, in which (R)ₙ is 6-methyl, R² is methyl, R³ and R⁴ are each hydrogen and R¹ for a compound corresponds in each case to one row of table A.

Table 262

Compounds of the formula I.Bb, in which (R)ₙ is 2,6-dichloro, R² is methyl, R³ and R⁴ are each hydrogen and R¹ for a compound corresponds in each case to one row of table A.

Table 263

Compounds of the formula I.Bb, in which (R)ₙ is 2,6-difluoro, R² is methyl, R³ and R⁴ are each hydrogen and R¹ for a compound corresponds in each case to one row of table A.

Table 264

Compounds of the formula I.Bb, in which (R)ₙ is 2,6-dimethyl, R² is methyl, R³ and R⁴ are each hydrogen, and R¹ for a compound corresponds in each case to one row of table A.

Table 265

Compounds of the formula I.Bb, in which (R)ₙ is 3-methyl-6-chloro, R² is methyl, R³ and R⁴ are each hydrogen and R¹ for a compound corresponds in each case to one row of table A.

Table 266

Compounds of the formula I.Bb, in which (R)ₙ is 3-methyl-6-fluoro, R² is methyl, R³ and R⁴ are each hydrogen and R¹ for a compound corresponds in each case to one row of table A.

Table 267

Compounds of the formula I.Bb, in which (R)ₙ is 3,6-dimethyl, R² is methyl, R³ and R⁴ are each hydrogen and R¹ for a compound corresponds in each case to one row of table A.

Table 268

Compounds of the formula I.Bb, in which (R)ₙ is 2-fluoro, R² is methyl, R³ and R⁴ are each hydrogen and R¹ for a compound corresponds in each case to one row of table A.

Table 269

Compounds of the formula I.Bb, in which (R)ₙ is 2-fluoro-6-chloro, R² is methyl, R³ and R⁴ are each hydrogen and R¹ for a compound corresponds in each case to one row of table A.

Table 270

Compounds of the formula I.Bb, in which (R)ₙ is 2-fluoro-methyl, R² is methyl, R³ and R⁴ are each hydrogen and R¹ for a compound corresponds in each case to one row of table A.

Table 271

Compounds of the formula I.Bb, in which (R)ₙ is 2,4-difluoro, R² is methyl, R³ and R⁴ are each hydrogen and R¹ for a compound corresponds in each case to one row of table A.

Table 272

Compounds of the formula I.Bb, in which (R)ₙ is 2,4,6-trifluoro, R² is methyl, R³ and R⁴ are each hydrogen and R¹ for a compound corresponds in each case to one row of table A.

Table 273

Compounds of the formula I.Bb, in which (R)ₙ is 2,4-difluoro-&chloro, R² is methyl, R³ and R⁴ are each hydrogen and R¹ for a compound corresponds in each case to one row of table A.

Table 274

Compounds of the formula I.Bb, in which (R)ₙ is 2,4-difluoro-6-methyl, R² is methyl, R³ and R⁴ are each hydrogen and R¹ for a compound corresponds in each case to one row of table A.

Table 275

Compounds of the formula I.Bb, in which (R)ₙ is 2-fluoro4-chloro, R² is methyl, R³ and R⁴ are each hydrogen and R¹ for a compound corresponds in each case to one row of table A.

Table 276

Compounds of the formula I.Bb, in which (R)ₙ is 2,6difluoro-4-chloro, R² is methyl, R³ and R⁴ are each hydrogen and R¹ for a compound corresponds in each case to one row of table A.

Table 277

Compounds of the formula I.Bb, in which (R)ₙ is 2-fluoro-4,6-dichloro, R² is methyl, R³ and R⁴ are each hydrogen and R¹ for a compound corresponds in each case to one row of table A.

Table 278

Compounds of the formula I.Bb, in which (R)ₙ is 2-fluoro-4-chloro-6-methyl, R² is methyl, R³ and R⁴ are each hydrogen and R¹ for a compound corresponds in each case to one row of table A.

Table 279

Compounds of the formula I.Bb, in which (R)ₙ is 2-fluoro-4-methyl, R² is methyl, R³ and R⁴ are each hydrogen and R¹ for a compound corresponds in each case to one row of table A.

Table 280

Compounds of the formula I.Bb, in which (R)ₙ is 2,6-difluoro-4-methyl, R² is methyl, R³ and R⁴ are each hydrogen and R¹ for a compound corresponds in each case to one row of table A.

Table 281

Compounds of the formula I.Bb, in which (R)ₙ is 2-fluoro4-methyl-6-chloro, R² is methyl, R³ and R⁴ are each hydrogen and R¹ for a compound corresponds in each case to one row of table A.

Table 282

Compounds of the formula I.Bb, in which (R)ₙ is 2-fluoro-4,6-dimethyl, R² is methyl, R³ and R⁴ are each hydrogen and R¹ for a compound corresponds in each case to one row of table A.

Table 283

Compounds of the formula I.Bb, in which (R)ₙ is 2-fluoro-4-methoxy, R² is methyl, R³ and R⁴ are each hydrogen and R¹ for a compound corresponds in each case to one row of table A.

Table 284

Compounds of the formula I.Bb, in which (R)ₙ is 2,6-difluoro-4-methoxy, R² is methyl, R³ and R⁴ are each hydrogen and R¹ for a compound corresponds in each case to one row of table A.

Table 285

Compounds of the formula I.Bb, in which (R)ₙ is 2-fluoro4-methoxy-6-chloro, R² is methyl, R³ and R¹ are each hydrogen and R¹ for a compound corresponds in each case to one row of table A.

Table 286

Compounds of the formula I.Bb, in which (R)ₙ is 2-fluoro4-methoxy-6-methyl, R² is methyl, R³ and R⁴ are each hydrogen and R¹ for a compound corresponds in each case to one row of table A.

Table 287

Compounds of the formula I.Bb, in which (R)ₙ is 2-fluoro-4-cyano, R² is methyl, R³ and R⁴ are each hydrogen and R¹ for a compound corresponds in each case to one row of table A.

Table 288

Compounds of the formula I.Bb, in which (R)ₙ is 2,6-difluoro-4cyano, R² is methyl, R³ and R⁴ are each hydrogen and R¹ for a compound corresponds in each case to one row of table A.

Table 289

Compounds of the formula I.Bb, in which (R)ₙ is 2-fluoro-4-cyano-6-chloro, R² is methyl, R³ and R⁴ are each hydrogen and R¹ for a compound corresponds in each case to one row of table A.

Table 290

Compounds of the formula I.Bb, in which (R)ₙ is 2-fluoro4-cyano-6-methyl, R² is methyl, R³ and R⁴ are each hydrogen and R¹ for a compound corresponds in each case to one row of table A.

Table 291

Compounds of the formula I.Bb, in which (R)ₙ is 2-chloro, R² is methyl, R³ and R⁴ are each hydrogen and R¹ for a compound corresponds in each case to one row of table A.

Table 292

Compounds of the formula I.Bb, in which (R)ₙ is 2-chloro-6-fluoro, R² is methyl, R³ and R⁴ are each hydrogen and R¹ for a compound corresponds in each case to one row of table A.

Table 293

Compounds of the formula I.Bb, in which (R)ₙ is 2-chloro-6-methyl, R² is methyl, R³ and R⁴ are each hydrogen and R¹ for a compound corresponds in each case to one row of table A.

Table 294

Compounds of the formula I.Bb, in which (R)ₙ is 2-chloro-4-fluoro, R² is methyl, R³ and R⁴ are each hydrogen and R¹ for a compound corresponds in each case to one row of table A.

Table 295

Compounds of the formula I.Bb, in which (R)ₙ is 2-chloro-4,6-difluoro, R² is methyl, R³ and R⁴ are each hydrogen and R¹ for a compound corresponds in each case to one row of table A.

Table 296

Compounds of the formula I.Bb, in which (R)ₙ is 2,6-dichloro-4-fluoro, R² is methyl, R³ and R⁴ are each hydrogen and R¹ for a compound corresponds in each case to one row of table A.

Table 297

Compounds of the formula I.Bb, in which (R)ₙ is 2-chloro-4-fluoro-6-methyl, R² is methyl, R³ and R⁴ are each hydrogen and R¹ for a compound corresponds in each case to one row of table A.

Table 298

Compounds of the formula I.Bb, in which (R)ₙ is 2,4-dichloro, R² is methyl, R³ and R⁴ are each hydrogen and R¹ for a compound corresponds in each case to one row of table A.

Table 299

Compounds of the formula I.Bb, in which (R)ₙ is 2,4-dichloro-6-fluoro, R² is methyl, R³ and R⁴ are each hydrogen and R¹ for a compound corresponds in each case to one row of table A.

Table 300

Compounds of the formula I.Bb, in which (R)ₙ is 2,4,6-trichloro, R² is methyl, R³ and R⁴ are each hydrogen and R¹ for a compound corresponds in each case to one row of table A.

Table 301

Compounds of the formula I.Bb, in which (R)ₙ is 2,4-dichloro-6-methyl, R² is methyl, R³ and R⁴ are each hydrogen and R¹ for a compound corresponds in each case to one row of table A.

Table 302

Compounds of the formula I.Bb, in which (R)ₙ is 2-chloromethyl, R² is methyl, R³ and R⁴ are each hydrogen and R¹ for a compound corresponds in each case to one row of table A.

Table 303

Compounds of the formula I.Bb, in which (R)ₙ is 2-chloro4-methyl-6-fluoro, R² is methyl, R³ and R⁴ are each hydrogen and R¹ for a compound corresponds in each case to one row of table A.

Table 304

Compounds of the formula I.Bb, in which (R)ₙ is 2,6-dichloro4methyl, R² is methyl, R³ and R⁴ are each hydrogen and R¹ for a compound corresponds in each case to one row of table A.

Table 305

Compounds of the formula I.Bb, in which (R)ₙ is 2-chloro4,6-dimethyl, R² is methyl, R³ and R⁴ are each hydrogen and R¹ for a compound corresponds in each case to one row of table A.

Table 306

Compounds of the formula I.Bb, in which (R)ₙ is 2-chloromethoxy, R² is methyl, R³ and R⁴ are each hydrogen and R¹ for a compound corresponds in each case to one row of table A.

Table 307

Compounds of the formula I.Bb, in which (R)ₙ is 2-chloro4methoxy-6-fluoro, R² is methyl, R³ and R⁴ are each hydrogen and R¹ for a compound corresponds in each case to one row of table A.

Table 308

Compounds of the formula I.Bb, in which (R)ₙ is 2,6-dichloro-4-methoxy, R² is methyl, R³ and R⁴ are each hydrogen and R¹ for a compound corresponds in each case to one row of table A.

Table 309

Compounds of the formula I.Bb, in which (R)ₙ is 2-chloro4-methoxy-6-methyl, R² is methyl, R³ and R⁴ are each hydrogen and R¹ for a compound corresponds in each case to one row of table A.

Table 310

Compounds of the formula I.Bb, in which (R)ₙ is 2-chloro-cyano, R² is methyl, R³ and R⁴ are each hydrogen and R¹ for a compound corresponds in each case to one row of table A.

Table 311

Compounds of the formula I.Bb, in which (R)ₙ is 2-chloro4-cyano-6-fluoro, R² is methyl, R³ and R⁴ are each hydrogen and R¹ for a compound corresponds in each case to one row of table A.

Table 312

Compounds of the formula I.Bb, in which (R)ₙ is 2,6dichloro4cyano, R² is methyl, R³ and R⁴ are each hydrogen and R¹ for a compound corresponds in each case to one row of table A.

Table 313

Compounds of the formula I.Bb, in which (R)ₙ is 2-chloro4-cyano-6-methyl, R² is methyl, R³ and R⁴ are each hydrogen and R¹ for a compound corresponds in each case to one row of table A.

Table 314

Compounds of the formula I.Bb, in which (R)ₙ is 2-methyl, R² is methyl, R³ and R⁴ are each hydrogen and R¹ for a compound corresponds in each case to one row of table A.

Table 315

Compounds of the formula I.Bb, in which (R)ₙ is 2-methyl-6-fluoro, R² is methyl, R³ and R⁴ are each hydrogen and R¹ for a compound corresponds in each case to one row of table A.

Table 316

Compounds of the formula I.Bb, in which (R)ₙ is 2-methyl-6-chloro, R² is methyl, R³ and R⁴ are each hydrogen and R¹ for a compound corresponds in each case to one row of table A

Table 317

Compounds of the formula I.Bb, in which (R)ₙ is 2-methyl-4-fluoro, R² is methyl, R³ and R⁴ are each hydrogen and R¹ for a compound corresponds in each case to one row of table A.

Table 318

Compounds of the formula I.Bb, in which (R)ₙ is 2-methyl4,6-difluoro, R² is methyl, R³ and R⁴ are each hydrogen and R¹ for a compound corresponds in each case to one row of table A.

Table 319

Compounds of the formula I.Bb, in which (R)ₙ is 2-methyl-4-fluoro-6-chloro, R² is methyl, R³ and R⁴ are each hydrogen and R¹ for a compound corresponds in each case to one row of table A.

Table 320

Compounds of the formula I.Bb, in which (R)ₙ is 2,6-dimethyl-4-fluoro, R² is methyl, R³ and R⁴ are each hydrogen and R¹ for a compound corresponds in each case to one row of table A.

Table 321

Compounds of the formula I.Bb, in which (R)ₙ is 2-methyl-4-chloro, R² is methyl, R³ and R⁴ are each hydrogen and R¹ for a compound corresponds in each case to one row of table A.

Table 322

Compounds of the formula I.Bb, in which (R)ₙ is 2-methyl-4-chloro-6-fluoro, R² is methyl, R³ and R⁴ are each hydrogen and R¹ for a compound corresponds in each case to one row of table A.

Table 323

Compounds of the formula I.Bb, in which (R)ₙ is 2-methyl-4,6-dichloro, R² is methyl, R³ and R⁴ are each hydrogen and R¹ for a compound corresponds in each case to one row of table A.

Table 324

Compounds of the formula I.Bb, in which (R)ₙ is 2,6-dimethyl-4-chloro, R² is methyl, R³ and R⁴ are each hydrogen and R¹ for a compound corresponds in each case to one row of table A.

Table 325

Compounds of the formula I.Bb, in which (R)ₙ is 2,4-dimethyl, R² is methyl, R³ and R⁴ are each hydrogen and R¹ for a compound corresponds in each case to one row of table A.

Table 326

Compounds of the formula I.Bb, in which (R)ₙ is 2,4-dimethyl-6-fluoro, R² is methyl, R³ and R⁴ are each hydrogen and R¹ for a compound corresponds in each case to one row of table A.

Table 327

Compounds of the formula I.Bb, in which (R)ₙ is 2,4-dimethyl-6-chloro, R² is methyl, R³ and R⁴ are each hydrogen and R¹ for a compound corresponds in each case to one row of table A.

Table 328

Compounds of the formula I.Bb, in which (R)ₙ is 2,4,6-trimethyl, R² is methyl, R³ and R⁴ are each hydrogen and R¹ for a compound corresponds in each case to one row of table A.

Table 329

Compounds of the formula I.Bb, in which (R)ₙ is 2-methyl-4-methoxy, R² is methyl, R³ and R⁴ are each hydrogen and R¹ for a compound corresponds in each case to one row of table A.

Table 330

Compounds of the formula I.Bb, in which (R)ₙ is 2-methyl-4-methoxy-6-fluoro, R² is methyl, R³ and R⁴ are each hydrogen and R¹ for a compound corresponds in each case to one row of table A.

Table 331

Compounds of the formula I.Bb, in which (R)ₙ is 2-methyl-4-methoxy-6-chloro, R² is methyl, R³ and R⁴ are each hydrogen and R¹ for a compound corresponds in each case to one row of table A.

Table 332

Compounds of the formula I.Bb, in which (R)ₙ is 2,6-dimethyl-4-methoxy, R² is methyl, R³ and R⁴ are each hydrogen and R¹ for a compound corresponds in each case to one row of table A.

Table 333

Compounds of the formula I.Bb, in which (R)ₙ is 2-methyl-4-cyano, R² is methyl, R³ and R⁴ are each hydrogen and R¹ for a compound corresponds in each case to one row of table A.

Table 334

Compounds of the formula I.Bb, in which (R)ₙ is 2-methyl4cyano-6-fluoro, R² is methyl, R³ and R⁴ are each hydrogen and R¹ for a compound corresponds in each case to one row of table A.

Table 335

Compounds of the formula I.Bb, in which (R)ₙ is 2-methylcyano-6-chloro, R² is methyl, R³ and R⁴ are each hydrogen and R¹ for a compound corresponds in each case to one row of table A.

Table 336

Compounds of the formula I.Bb, in which (R)ₙ is 2,6-dimethyl4-cyano, R² is methyl, R³ and R⁴ are each hydrogen and R¹ for a compound corresponds in each case to one row of table A.

Table 337

Compounds of the formula I.Bb, in which (R)ₙ is 2-chloro, R² is methyl, R³ and R⁴ are each methyl and R¹ for a compound corresponds in each case to one row of table A.

Table 338

Compounds of the formula I.Bb, in which (R)ₙ is 2-fluoro, R² is methyl, R³ and R⁴ are each methyl and R¹ for a compound corresponds in each case to one row of table A.

Table 339

Compounds of the formula I.Bb, in which (R)ₙ is 2-methyl, R² is methyl, R³ and R⁴ are each methyl and R¹ for a compound corresponds in each case to one row of table A.

Table 340

Compounds of the formula I.Bb, in which (R)ₙ is 6-chloro, R² is methyl, R³ and R⁴ are each methyl and R¹ for a compound corresponds in each case to one row of table A.

Table 341

Compounds of the formula I.Bb, in which (R)ₙ is 6-fluoro, R² is methyl, R³ and R⁴ are each methyl and R¹ for a compound corresponds in each case to one row of table A.

Table 342

Compounds of the formula I.Bb, in which (R)ₙ is 6-methyl, R² is methyl, R³ and R⁴ are each methyl and R¹ for a compound corresponds in each case to one row of table A.

Table 343

Compounds of the formula I.Bb, in which (R)ₙ is 2,6-dichloro, R² is methyl, R³ and R⁴ are each methyl and R¹ for a compound corresponds in each case to one row of table A.

Table 344

Compounds of the formula I.Bb, in which (R)ₙ is 2,6-difluoro, R² is methyl, R³ and R⁴ are each methyl and R¹ for a compound corresponds in each case to one row of table A.

Table 345

Compounds of the formula I.Bb, in which (R)ₙ is 2,6-dimethyl, R² is methyl, R³ and R⁴ are each methyl and R¹ for a compound corresponds in each case to one row of table A.

Table 346

Compounds of the formula I.Bb, in which (R)ₙ is 2,6-dichloro, R² is methyl, R³ and R⁴ are each methyl and R¹ for a compound corresponds in each case to one row of table A.

Table 347

Compounds of the formula I.Bb, in which (R)ₙ is 3-methyl-chloro, R² is methyl, R³ and R⁴ are each methyl and R¹ for a compound corresponds in each case to one row of table A.

Table 348

Compounds of the formula I.Bb, in which (R)ₙ is 3-methyl-6-fluoro, R² is methyl, R³ and R⁴ are each methyl and R¹ for a compound corresponds in each case to one row of table A.

Table 349

Compounds of the formula I.Bb, in which (R)ₙ is 3,6-dimethyl, R² is methyl, R³ and R⁴ are each methyl and R¹ for a compound corresponds in each case to one row of table A.

Table 350

Compounds of the formula I.Bb, in which (R)ₙ is 2-fluoro-&-chloro, R² is methyl, R³ and R⁴ are each methyl and R¹ for a compound corresponds in each case to one row of table A.

Table 351

Compounds of the formula I.Bb, in which (R)ₙ is 2-fluoro-6-methyl, R² is methyl, R³ and R⁴ are each methyl and R¹ for a compound corresponds in each case to one row of table A.

Table 352

Compounds of the formula I.Bb, in which (R)ₙ is 2-chloro-6-fluoro, R² is methyl, R³ and R⁴ are each methyl and R¹ for a compound corresponds in each case to one row of table A.

Table 353

Compounds of the formula I.Bb, in which (R)ₙ is 2-chloro-6-methyl, R² is methyl, R³ and R⁴ are each methyl and R¹ for a compound corresponds in each case to one row of table A.

Table 354

Compounds of the formula I.Bb, in which (R)ₙ is 2-chloro, R² is methyl, R³ is hydrogen, R⁴ is methyl and R¹ for a compound corresponds in each case to one row of table A.

Table 355

Compounds of the formula I.Bb, in which (R)ₙ is 2-fluoro, R² is methyl, R³ is hydrogen, R⁴ is methyl and R¹ for a compound corresponds in each case to one row of table A.

Table 356

Compounds of the formula I.Bb, in which (R)ₙ is 2-methyl, R² is methyl, R³ and R⁴ are each hydrogen and R¹ for a compound corresponds in each case to one row of table A.

Table 357

Compounds of the formula I.Bb, in which (R)ₙ is 6-chloro, R² is methyl, R³ is hydrogen, R⁴ is methyl and R¹ for a compound corresponds in each case to one row of table A.

Table 358

Compounds of the formula I.Bb, in which (R)ₙ is 6-fluoro, R² is methyl, R³ is hydrogen, R⁴ is methyl and R¹ for a compound corresponds in each case to one row of table A.

Table 359

Compounds of the formula I.Bb, in which (R)ₙ is 6-methyl, R² is methyl, R³ is hydrogen, R⁴is methyl and R¹ for a compound corresponds in each case to one row of table A.

Table 360

Compounds of the formula I.Bb, in which (R)ₙ is 2,6-dichloro, R² is methyl, R³ is hydrogen, R⁴ is methyl and R¹ for a compound corresponds in each case to one row of table A.

Table 361

Compounds of the formula I.Bb, in which (R)ₙ is 2,6-difluoro, R² is methyl, R³ is hydrogen, R⁴ is methyl and R¹ for a compound corresponds in each case to one row of table A.

Table 362

Compounds of the formula I.Bb, in which (R)ₙ is 2,6-dimethyl, R² is methyl, R³ is hydrogen, R⁴ is methyl and R¹ for a compound corresponds in each case to one row of table A.

Table 363

Compounds of the formula I.Bb, in which (R)ₙ is 2,6-dichloro, R² is methyl, R³ is hydrogen, R⁴ is methyl and R¹ for a compound corresponds in each case to one row of table A.

Table 364

Compounds of the formula I.Bb, in which (R)ₙ is 3-methyl-6-chloro, R² is methyl, R³ is hydrogen, R⁴ is methyl and R¹ for a compound corresponds in each case to one row of table A.

Table 365

Compounds of the formula I.Bb, in which (R)ₙ is 3-methyl-6-fluoro, R² is methyl, R³ is hydrogen, R⁴ is methyl and R¹ for a compound corresponds in each case to one row of table A.

Table 366

Compounds of the formula I.Bb, in which (R)ₙ is 3,6-dimethyl, R² is methyl, R³ is hydrogen, R⁴ is methyl and R¹ for a compound corresponds in each case to one row of table A.

Table 367

Compounds of the formula I.Bb, in which (R)ₙ is 2-fluoro-6-chloro, R² is methyl, R³ is hydrogen, R⁴ is methyl and R¹ for a compound corresponds in each case to one row of table A.

Table 368

Compounds of the formula I.Bb, in which (R)ₙ is 2-fluoro-6-methyl, R² is methyl, R³ is hydrogen, R⁴ is methyl and R¹ for a compound corresponds in each case to one row of table A.

Table 369

Compounds of the formula I.Bb, in which (R)ₙ is 2-chloro-6-fluoro, R² is methyl, R³ is hydrogen, R⁴ is methyl and R¹ for a compound corresponds in each case to one row of table A.

Table 370

Compounds of the formula I.Bb, in which (R)ₙ is 2-chloro-6-methyl, R² is methyl, R³ is hydrogen, R⁴ is methyl and R¹ for a compound corresponds in each case to one row of table A.

Table 371

Compounds of the formula I.Bb, in which (R)ₙ is 2-chloro, R² is methyl, R³ is hydrogen, R⁴ is isopropyl and R¹ for a compound corresponds in each case to one row of table A.

Table 372

Compounds of the formula I.Bb, in which (R)ₙ is 2-fluoro, R² is methyl, R³ is hydrogen, R⁴ is isopropyl and R¹ for a compound corresponds in each case to one row of table A.

Table 373

Compounds of the formula I.Bb, in which (R)ₙ is 2-methyl, R² is methyl, R³ is hydrogen, R⁴ is isopropyl and R¹ for a compound corresponds in each case to one row of table A.

Table 374

Compounds of the formula I.Bb, in which (R)ₙ is 6-chloro, R² is methyl, R³ is hydrogen, R⁴ is isopropyl and R¹ for a compound corresponds in each case to one row of table A.

Table 375

Compounds of the formula I.Bb, in which (R)ₙ is 6-fluoro, R² is methyl, R³ is hydrogen, R⁴ is isopropyl and R¹ for a compound corresponds in each case to one row of table A.

Table 376

Compounds of the formula I.Bb, in which (R)ₙ is 6-methyl, R² is methyl, R³ is hydrogen, R⁴ is isopropyl and R¹ for a compound corresponds in each case to one row of table A.

Table 377

Compounds of the formula I.Bb, in which (R)ₙ is 2,6-dichloro, R² is methyl, R³ is hydrogen, R⁴ is isopropyl and R¹ for a compound corresponds in each case to one row of table A.

Table 378

Compounds of the formula I.Bb, in which (R)ₙ is 2,6-difluoro, R²is methyl, R³ is hydrogen, R⁴ is isopropyl and R¹ for a compound corresponds in each case to one row of table A.

Table 379

Compounds of the formula I.Bb, in which (R)ₙ is 2,6-dimethyl, R² is methyl, R³ is hydrogen, R⁴ is isopropyl and R¹ for a compound corresponds in each case to one row of table A.

Table 380

Compounds of the formula I.Bb, in which (R)ₙ is 2,6-dichloro, R² is methyl, R³ is hydrogen, R⁴ is isopropyl and R¹ for a compound corresponds in each case to one row of table A.

Table 381

Compounds of the formula I.Bb, in which (R)ₙ is 3-methyl-6-chloro, R² is methyl, R³ is hydrogen, R⁴ is isopropyl and R¹ for a compound corresponds in each case to one row of table A.

Table 382

Compounds of the formula I.Bb, in which (R)ₙ is 3-methyl-6-fluoro, R² is methyl, R³ is hydrogen, R⁴ is isopropyl and R¹ for a compound corresponds in each case to one row of table A.

Table 383

Compounds of the formula I.Bb, in which (R)ₙ is 3,6-dimethyl, R² is methyl, R³ is hydrogen, R⁴ is isopropyl and R¹ for a compound corresponds in each case to one row of table A.

Table 384

Compounds of the formula I.Bb, in which (R)ₙ is 2-fluoro-6-chloro, R² is methyl, R³ is hydrogen, R⁴ is isopropyl and R¹ for a compound corresponds in each case to one row of table A.

Table 385

Compounds of the formula I.Bb, in which (R)ₙ is 2-fluoro-6-methyl, R² is methyl, R³ is hydrogen, R⁴ is isopropyl and R¹ for a compound corresponds in each case to one row of table A.

Table 386

Compounds of the formula I.Bb, in which (R)ₙ is 2-chloro-6-fluoro, R² is methyl, R³ is hydrogen, R⁴ is isopropyl and R¹ for a compound corresponds in each case to one row of table A.

Table 387

Compounds of the formula I.Bb, in which (R)ₙ is 2-chloro-6-methyl, R² is methyl, R³ is hydrogen, R⁴ is isopropyl and R¹ for a compound corresponds in each case to one row of table A.

Compounds I in which X is nitro can be obtained by different routes; advantageously, the starting material used is 5-aminotriazole of the formula II which is condensed with dicarbonyl compounds of the formula III.

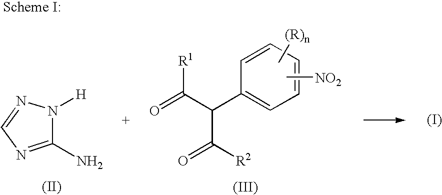

In scheme I, the substituents R¹, R², R and the index n are as defined above.

This reaction is usually carried out at temperatures of from 80° C. to 250° C., preferably from 120° C. to 180° C., without solvent or in an inert organic solvent in the presence of a base [cf. EP-A 770 615] or in the presence of acetic acid under the conditions known from Adv. Het. Chem. 57 (1993), 81 ff.

Suitable solvents are aliphatic carboxylic acids, for example C₁-C₄-carboxylic acids, such as acetic acid or propionic acid, aliphatic hydrocarbons, aromatic hydrocarbons, such as toluene, o-, m- and p-xylene, halogenated hydrocarbons, ethers, nitriles, ketones, alcohols, and also N-methylpyrrolidone, dimethyl sulfoxide, dimethylformamide and dimethylacetamide. Particularly preferably, the reaction is carried out without solvent or in acetic acid, propionic acid, ethylene glycol dimethyl ether, chlorobenzene, xylene, dimethyl sulfoxide, N-methylpyrrolidone. It is also possible to use mixtures of the solvents mentioned.

Suitable bases are, in general, inorganic compounds, such as alkali metal and alkaline earth metal hydroxides, alkali metal and alkaline earth metal oxides, alkali metal and alkaline earth metal hydrides, alkali metal amides, alkali metal and alkaline earth metal carbonates, and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, diisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to tertiary amines, such as diisopropylethylamine, tributylamine, N-methylmorpholine or N-methylpiperidine.

The bases are generally employed in catalytic amounts; however, they can also be employed in equimolar amounts, in excess or, if appropriate, as solvent.

The starting materials are generally reacted with one another in equimolar amounts. In terms of yield, it may be advantageous to employ an excess of base and diketone III, based on II.

The diketones III can be prepared analogously to processes known from the literature, for example as described in the publications mentioned above.

Compounds of the formula I in which X is NO₂ and R¹, R², R and n are as defined above can also, as shown in scheme 2, be obtained by coupling 5-halotriazolo-pyrimidines of the formula IV (Y is halogen, in particular chlorine or bromine) with organometallic reagents of the formula V.

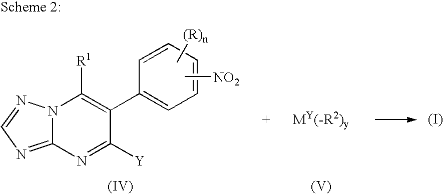

In formula V, M is a metal ion of valency y, such as, for example, B, Zn, Mg or Sn. In one embodiment of this process, the reaction is carried out with transition metal catalysis, such as Ni or Pd catalysis. This reaction can be carried out, for example, analogously to the following methods: J. Chem. Soc. Perkin Trans. 1 (1994), 1187, ibid. 1 (1996), 2345; WO-A 99/41255; Aust. J. Chem. 43 (1990), 733; J. Org. Chem. 43 (1978), 358; J. Chem. Soc. Chem. Commun. (1979), 866; Tetrahedron Lett. 34 (1993), 8267; ibid. 33 (1992), 413. The reaction may also be carried out without catalysts, in particular if M is Zn or Mg. The compounds IV are known from the publications cited at the outset. They are obtained, in particular, from 5,7-dichlorotriazolopyrimidines by introducing the radical R¹ using organometallic processes similar to those described above.

The compounds of the formula I according to the invention in which X is nitro are also obtainable by reacting 5-halotriazolopyrimidines of the formula IV with substituted malonic esters of the formula VI in which R^{x} is C₁-C₄-alkyl, allyl, phenyl or benzyl, subsequent hydrolysis of the ester VII formed and decarboxylation of the carboxylic acid VIIa, as shown in scheme 3.

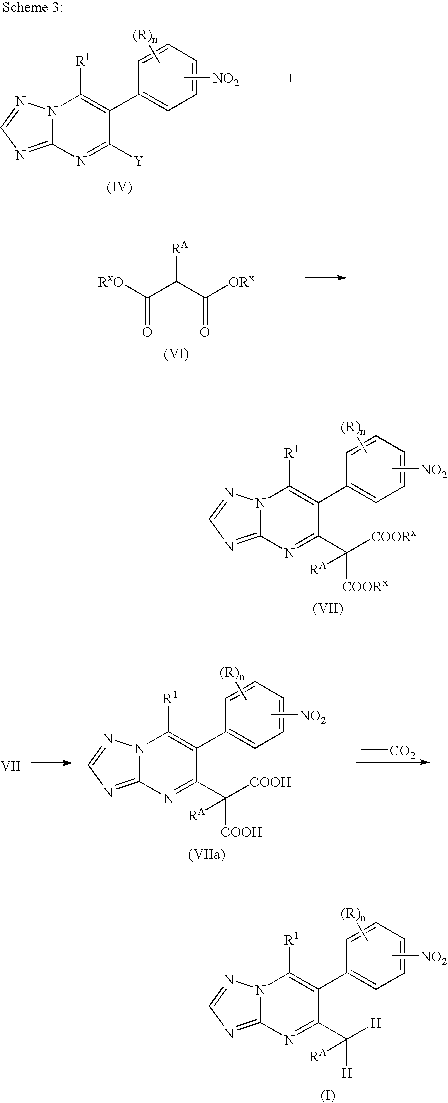

In scheme 3, Y is halogen, in particular chlorine or bromine, n, R and R¹ are as defined for formula I and R^{A} is hydrogen or C₁-C₃-alkyl which may be substituted by halogen, cyano, nitro or C₁-C₂-alkoxy. The nitro compounds IV are known from the publications cited at the outset.

In a preferred embodiment of the process according to the invention, R^{A} is hydrogen or methyl, in particular hydrogen.

The reaction is usually carried out in the presence of an inert organic solvent. Examples of suitable organic inert solvents are those mentioned above, in particular nitriles, such as acetonitrile. Compound VI is usually employed in an equimolar amount or in up to 4-fold excess, based on the nitro compounds IV.

The starting materials VI are known from the literature [J. Am. Chem. Soc. 64 (1942), 2714; J. Org. Chem. 39 (1974), 2172; Helv. Chim. Acta 61 (1978), 1565], or they can be prepared in accordance with the literature cited.

The subsequent hydrolysis of the ester VII is carried out under generally customary conditions [cf.: Greene & Wuts, Protective Groups in Organic Synthesis, Wiley (1991), p. 224 ff.: cleavage of alkyl esters under Pd catalysis (p. 248); hydrogenolysis of benzyl esters (p. 251); cleavage of methyl or ethyl esters in the presence of lithium salts such as Lil (p. 232), LiBr or LiCl; or under acidic or alkaline conditions]. Depending on the structural elements R^{A}, (R)ₙ and R¹, alkaline or acidic hydrolysis of the compounds VII may be advantageous. Under the conditions of ester hydrolysis, there may already be complete or partial decarboxylation to I.

The decarboxylation is usually carried out at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitrites, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; particularly preferably, the reaction is carried out in hydrochloric acid or acetic acid. It is also possible to use mixtures of the solvents mentioned.

The compounds of the formula I according to the invention in which X is nitro can also be obtained by nitrating the compounds of the formula VIII, as shown in scheme 4.

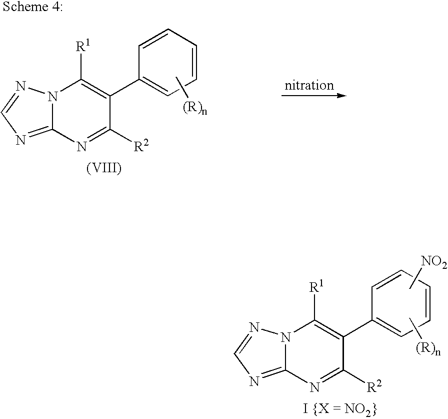

In scheme 4, R¹, R², R and n are as defined above. Suitable nitrating agents are, for example, nitric acid in various concentrations, including concentrated and fuming nitric acid, mixtures of sulfuric acid and nitric acid, and furthermore acetyl nitrates and alkyl nitrates.

The reaction can be carried out either in the absence of a solvent in an excess of the nitrating agent, or in an inert solvent or diluent, suitable solvents or diluents being, for example, water, mineral acids, organic acids, halogenated hydrocarbons, such as methylene chloride, anhydrides, such as acetic anhydride, and mixtures of these solvents.

The starting material VIII and the nitrating agent are expediently employed in approximately equimolar amounts; however, for an optimum conversion of the starting material it may be advantageous to use an excess of nitrating agent of up to about 10 times the molar amount, based on the starting material VIII. If the reaction is carried out without solvent in the nitrating agent, this is present in an even greater excess.

The reaction temperature is usually from −100° C. to 200° C., preferably from −30 to 50° C.

The starting materials VIII are known from WO 03/004465 or can be prepared analogously to the processes described therein.

The compounds of the formula I according to the invention in which X is C(S)NR³R⁴ can also be obtained by different routes, for example from the carboxamide compounds IX by reaction with a sulfurizing agent, as shown in scheme 5.

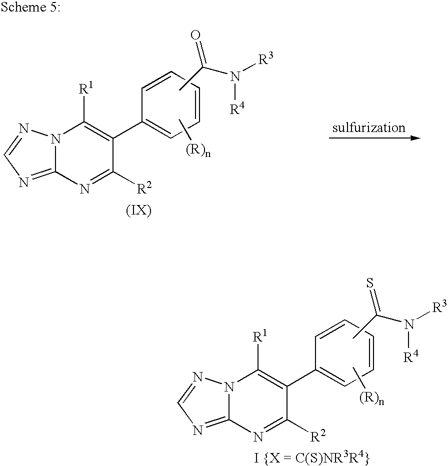

In scheme 5, R¹, R², R³, R⁴, R and n are as defined above. Examples of suitable sulfurizing agents are organophosphorus sulfides, such as Lawesson's reagent (2,2-bis-(4-methoxyphenyl)-1,3,2,4-dithiadiphosphetane 2,4-disulfide), organotin sulfides, such as bis(tricyclohexyltin)sulfide, or phosphorus pentasulfide (see also J. March, Advanced Organic Synthesis, 4th edition, Wiley Interscience 1992, p. 893 f. and the literature cited therein). The reaction can be carried out in a solvent or neat. Suitable solvents are the inert organic solvents mentioned above, and also pyridine and the like. The temperature required for the reaction is generally above room temperature and in particular in the range from 50 to 200° C.

The starting materials IX are known from WO 03/004465 or can be prepared analogously to the processes described therein. The starting materials IX can also be prepared according to the synthesis shown in scheme 6:

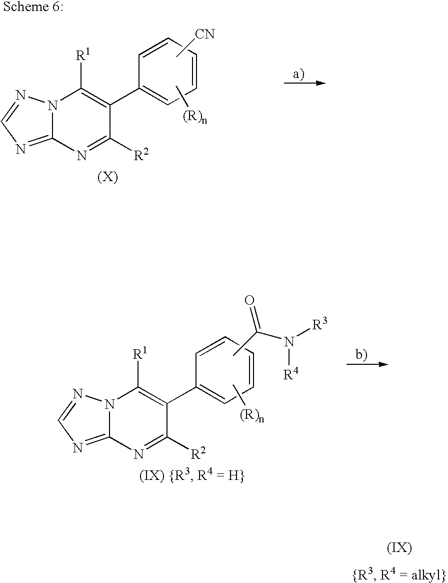

In scheme 6, R¹, R², R and n are as defined above.

In a first step a), a nitrile compound of the formula X is partially hydrolyzed. The hydrolysis of the nitrile X can be carried out either under acidic conditions, for example in the presence of a mineral acid, such as hydrochloric acid, or under alkaline conditions, for example in alkaline hydrogen peroxide solution. This gives a carboxamide IX in which R³ and R¹ are hydrogen.

The amide IX is then, if appropriate, mono- or dialkylated at the amide nitrogen atom in a second step b) by reaction with an alkylating agent. Suitable alkylating agents are, for example, C₁-C₆-alkyl halides, di-C₁-C₆-alkyl sulfates or C₁-C₆-alkyl phenylsulfonates, where the phenyl radical may, if appropriate, carry one or two radicals selected from the group consisting of nitro and C₁-C₆-alkyl. In general, an at least equimolar amount of alkylating agent, based on the acid amide IX, is employed. The alkylation is usually carried out in the presence of a base. Suitable bases are, in principle, all compounds which are capable of deprotonating the amide nitrogen. Suitable bases are, for example, alkali metal and alkaline earth metal hydroxides, such as sodium hydroxide, potassium hydroxide, lithium hydroxide and magnesium hydroxide, alkali metal and alkaline earth metal oxides, such as calcium oxide, alkali metal or alkaline earth metal carbonates, such as lithium carbonate, sodium carbonate, potassium carbonate, magnesium carbonate, calcium carbonate. Based on the acid amide IX, the base can be employed in substoichiometric, superstoichiometric or equimolar amounts.

The nitriles X are likewise known from WO 03/004465 or can be prepared from the compounds I in which X is nitro by reducing the nitro group to the amino group, conversion of the amino group into a diazonium group and subsequent Sandmeyer reaction with copper(I) cyanide.

The compounds of the formula I according to the invention in which X is C(═S)NH₂ can also be prepared, for example, by reacting the nitriles X with hydrogen sulfide, as shown in scheme 7.

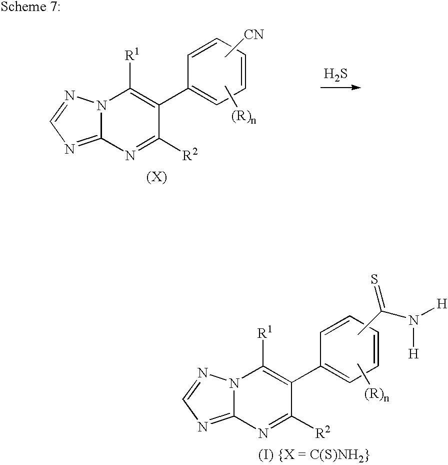

In scheme 7, (R)ₙ, R¹ and R² are as defined above. In general, the reaction is carried out in the presence of a solvent or diluent. Suitable solvents or diluents are, for example, aromatic amines, such as pyridine, substituted pyridines, such as collidine and lutidine, or tertiary amines, such as trimethylamine, triethylamine, triisopropylamine and N-methylpiperidine.

The reaction of the nitrile X with hydrogen sulfide is advantageously carried out at from 0° C. to 100° C., in particular from 10° C. to 50° C.

The aminothiocarbonylphenyltriazolopyrimidines I {X═—C(S)NH₂} obtained in this manner can then, if appropriate, be mono- or dialkylated at the amide nitrogen atom in a subsequent step, by reaction with an alkylating agent. Suitable alkylating agents are, for example, C₁-C₆-alkyl halides, di-C₁-C₆-alkyl sulfates or C₁-C₆-alkyl phenylsulfonates, where the phenyl radical may, if appropriate, carry one or two radicals selected from the group consisting of nitro and C₁-C₆-alkyl. In general, an at least equimolar amount of alkylating agent, based on the thioamide I, is employed.

The alkylation is usually carried out in the presence of a base. Suitable bases are, in principle, all compounds capable of deprotonating the amide nitrogen. Suitable bases are, for example, alkali metal and alkaline earth metal hydroxides, such as sodium hydroxide, potassium hydroxide, lithium hydroxide and magnesium hydroxide, alkali metal and alkaline earth metal oxides, such as calcium oxide, alkali metal or alkaline earth metal carbonates, such as lithium carbonate, sodium carbonate, potassium carbonate, magnesium carbonate, calcium carbonate. Based on the thioamide I, the base can be employed in substoichiometric, superstoichiometric or equimolar amounts.

Compounds of the formula I in which X is a group —C(═N—OR⁵)(NR⁶R⁷) can be prepared, for example, by reacting compounds of the formula I in which X is —C(S)NR³R⁴ with hydroxylamine hydrochloride followed, if appropriate, by alkylation. Here, R³ and R⁴ have the same meanings as R⁶ and R⁷. With respect to suitable alkylating agents, solvents and bases, reference is made to what was said above in its entirety.

Compounds of the formula I in which X is a group —C(═N—NR⁸R⁹)(NR¹⁰R¹¹) can be prepared, for example, by reacting compounds of the formula I in which X is —C(S)NR³R⁴ with substituted hydrazine derivatives. Here, R³ and R⁴ have the same meanings as R¹⁰ and R¹¹.

Compounds of the formula I in which X is a group —C(═N—OR⁵)(NH₂) or —C(═N—NR⁸R⁹)—NH₂ can advantageously also be prepared from the nitrile X using the methods shown in scheme 8. Here, the nitrile X may, firstly, be reacted directly with NH₂OR⁵ (where R⁵ is as defined above) or its salts in aqueous solution, preferably in water or water/alkanol mixtures, if appropriate in the presence of a base, to give the compound I where X═—C(═N—OR⁵)(NH₂), or with H₂N—NR⁸R⁹ to give the compound I where X═—C(═N—NR⁸R⁹)—NH₂. This reaction can be carried out, for example, analogously to the following methods: WO 00/17156, WO 00/24740, U.S. Pat. No. 5,104,991, U.S. Pat. No. 4,379,158, Journal of Organic Chemistry 58 (16) (1993), 4331, Acta Pol. Pharm. 36 (1979), 155. Secondly, compounds of the formula I in which X is a group —C(═N—OR⁵)(NH₂) or a group —C(═N—NR⁸R⁹)—NH₂ can also be prepared by reacting the nitrile X with an alcohol R¹²—OH, such as a C₁-C₄-alkanol, and hydrogen chloride in the absence of water to give an imidocarboxylic ester XI, followed by reaction of the resulting imidocarboxylic ester XI with NH₂OR⁵, giving the compound I where X═—C(═N—OR⁵)(NH₂), or with H₂N—NR⁸R⁹ (in which R⁵, R⁸ and R⁹ are as defined above), giving the compound I where X═—C(═N—NR⁸R⁹)—NH₂. The preparation of the imidocarboxylic ester XI can be carried out, for example, analogously to the conditions known from Jerry March, 3rd edition, John Wiley & Sons, New York, 1985, p. 792.

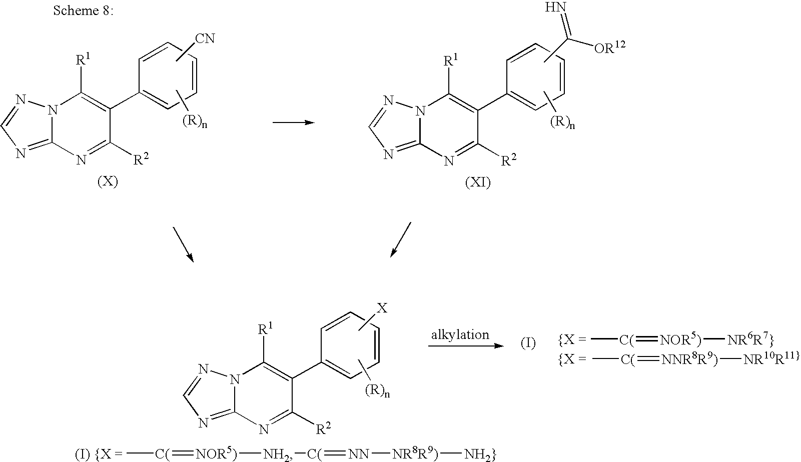

In scheme 8, R¹, R², (R)ₙ are as defined above; R¹² is, for example, C₁-C₄-alkyl. The resulting compounds I in which X is a group —C(═N—OR⁵)(NH₂) or —C(═N—NR⁸R⁹)NH₂ can be alkylated in a known manner giving compounds I in which X is —C(═N—OR⁵)(NR⁶R⁷) or —C(═N—NR⁸R⁹)(NR¹⁰R¹¹), where R⁵, R⁶, R⁷, R⁸, R⁹, R¹⁰ and R¹¹ are as defined above. With respect to suitable processes for the alkylation, reference is made to what was said above in its entirety.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is, however, generally not necessarily required since in some cases the individual isomers can be interconverted during work-up for use or during application (for example under the action of light, acids or bases). Such conversions may also take place after the application, for example in the treatment of plants, in the treated plant or in the harmful fungus or animal pests to be controlled.

The compounds I are suitable as fungicides. They are distinguished by an outstanding effectiveness against a broad spectrum of phytopathogenic fungi from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, corn, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pydiculatia oryzae on rice,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected against fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g per kilogram of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dustable products, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially.
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ethers, ethoxylated isooctylphenol, octyiphenol, nonylphenol, alkylphenol polyglycol ethers, tributylphenyl polyglycol ethers, tristearylphenyl polyglycol ethers, alkylaryl polyether alcohols, alcohol and fatty alcohovethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations:

1. Products for Dilution with Water

A) Water-Soluble Concentrates (SL)
10 parts by weight of a compound according to the invention are dissolved in water or in a water-soluble solvent. As an alternative, wetters or other auxiliaries are added. The active compound dissolves upon dilution with water.

B) Dispersible Concentrates (DC)
20 parts by weight of a compound according to the invention are dissolved in cyclohexanone with addition of a dispersant, for example polyvinylpyrrolidone.

Dilution with water gives a dispersion.

C) Emulsifiable Concentrates (EC)
15 parts by weight of a compound according to the invention are dissolved in xylene with addition of calcium dodecylbenzenesulfonate and castor oil ethoxylate (in each case 5%). Dilution with water gives an emulsion.

D) Emulsions (EW, EO)
40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.

E) Suspensions (SC, OD)
In an agitated ball mill, 20 parts by weight of a compound according to the invention are comminuted with addition of dispersants, wetters and water or an organic solvent to give a fine active compound suspension. Dilution with water gives a stable suspension of the active compound.

F) Water-Dispersible Granules and Water-Soluble Granules (WG, SG)
50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

G) Water-Dispersible Powders and Water-Soluble Powders (WP, SP)
75 parts by weight of a compound according to the invention are ground in a rotor-stator mill with addition of dispersants, wetters and silica gel. Dilution with water gives a stable dispersion or solution of the active compound.

2. Products to be Applied Undiluted

H) Dustable Powders (DP)
5 parts by weight of a compound according to the invention are ground finely and mixed intimately with 95% of finely divided kaolin. This gives a dustable product.

I) Granules (GR, FG, GG, MG)
0.5 part by weight of a compound according to the invention is ground finely and associated with 95.5% of carriers. Current methods are extrusion, spray-drying or the fluidized bed. This gives granules to be applied undiluted.

J) ULV Solutions (UL)
10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the compositions according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the use form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, mydozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, badinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthalisopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, toloclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolymfluanid,
cinnamides and analogous compounds, such as dimethomorph, flumetover or flumorph.

### SYNTHESIS EXAMPLES

### Example 1

### Preparation of 5-methyl-6-(2chloro-4-nitrophenyl)-7-(2-methylbutyl)-1,2,4-triazolo[1,5-a]pyrimidine

1.1 5-(Dimethylmalon-2-yl)-6-(2-chloro-4-nitrophenyl)-7-(2-methylbutyl)-1,2,4-triazolo-[1,5-a]pyrimidine

1 g (6.5 mmol) of sodium dimethylmalonate was added to 1 g (2.6 mol) of 5-chloro-6-(2-chloro4-nitrophenyl)-7-(2-methylbutyl)-1,2,4-triazolo[1,5-a]-pyrimidine (prepared as described in WO 99/41255) in 10 ml of acetonitrile, the mixture was stirred at 70 to 80° C. for 3 hours.

The reaction mixture was then cooled to room temperature, and the yellow precipitate was filtered off with suction. Dilute hydrochloric acid was added to the yellow precipitate, the mixture was stirred until it was decolorized (about 15 minutes) and the aqueous mixture was extracted with methyl tert-butyl ether. The combined organic phases were filtered off with suction through silica gel, and the filtrate was concentrated under reduced pressure, giving 0.9 g of (73% yield) of the title compound as a light yellow viscous material.

¹H-NMR (CDCl₃) δ: 8.6 (s, 1H), 8.5 (s, 1H), 8.3 (d, 1H), 7.55 (dd, 1H), 4.65 (2s, 1H), 3.8 (2s, 1H), 3.7 (2s, 1H), 3.1 (dd, 0.5H), 2.95 (dd, 0.5H), 2.7 (dd, 0.5H), 2.6 (dd, 0.5H), 2.1 (m, 1H), 1.3 (m, 1H), 1.05 (m, 1H), 0.7-0.9 (m, 6H).

1.2 5-Methyl-6-(2-chloro-4-nitrophenyl)-7-(2-methylbutyl)-1,2,4-triazolo[1,5-a]-pyrimidine

A mixture of 0.9 g (1.9 mmol) of 5-(dimethylmalon-2-yl)6-(2-chloro-4-nitrophenyly 7-(2-methylbutyl)-1,2,4-triazolo[1,5-a]pyrimidine from example 1.1 and 0.8 g (10 mmol) of 50% strength aqueous sodium hydroxide solution in 25 ml of methanol/water 4:1 was stirred at a bath temperature of 70° C. for 4 hours.

The mixture was then allowed to cool to room temperature and acidified with dilute hydrochloric acid, and the aqueous phase was extracted with methyl tert-butyl ether. The combined organic phases were washed with water, dried and concentrated under reduced pressure. The residue crystallized and was triturated with diisopropyl ether. This gave 0.35 g (60% yield) of the title compound as a colorless solid of melting point 158 to 162° C.

¹H-NMR (CDCl₃) δ: 8.5 (s, 2H), 8.35 (d, 1H), 7.55 (2d, 1H), 3.11 (dd, 0.4H), 2.9 (dd, 0.6H), 2.8 (dd, 0.6H), 2.6 (dd, 0.4H), 2.4 (s, 3H), 2.1 (m, 1H), 1.0-1.4 (m, 2H), 0.7-0.85 (m, 6H).

### Example 2

### Preparation of 5-methyl-6-(2-chloro-4-(aminothiocarbonyl)phenyl)-7-(2-methylbut-1 -yl)-[1,2,4]-triazolo[1,5-a]pyrimidine

At 35° C., hydrogen sulfide gas was introduced for 3 hours into a mixture of 0.5 g (1.5 mmol) of 5-methyl-6-(2-chloro-4-cyanophenyl)-7-(2-methylbut-1-yl)-[1,2,4]-triazolo[1,5-a]pyrimidine (prepared analogously to WO 03/04465) in 10 ml of pyridine, and the reaction mixture was then stirred at room temperature for 2.5 days. The reaction mixture was then diluted with diethyl ether and the organic phase was extracted with dilute hydrochloric acid. The organic phase was dried over magnesium sulfate and then concentrated under reduced pressure. The residue was purified by preparative MPLC on silica gel RP 18 using acetonitrile/water mixture. This gave 0.45 g (80% of theory) of the title compound as a yellow resin having a melting point of 173 to 177° C.

¹H-NMR (CDCl₃, δ in ppm): 8.45 (s, 1H); 8.2 (2s, 2H); 8.1 (s, broad, 1H); 8.05 (d, 1H); 7.35 (d, 1H); 3.1 (dd, 0.5H); 2.9 (dd, 0.5H); 2.85 (dd, 0.5H); 2.6 (dd, 0.5H); 2.4 (s, 3H); 2.1 (m, 1H); 1.0-1.4 (m, 2H); 0.8 (m, 6H).

Examples for the Action Against Harmful Fungi

The fungicidal action of the compounds of the formula I was demonstrated by the following tests:

The active compounds were prepared as a stock solution comprising 0.25% by weight of active compound in acetone or dimethyl sulfoxide (DMSO). 1% by weight of the emulsifier Uniperol® EL (wetting agent having emulsifying and dispersing action based on ethoxylated alkylphenols) was added to this solution, and the mixture was diluted with water to the desired concentration.

### Use Example 1

### Activity Against Early Blight of Tomato Caused by Altemara Solani

Leaves of potted plants of the cultivar “St. Pierre beef tomato” were sprayed to run off point with an aqueous suspension having the concentration of active compounds stated below. The next day, the leaves were infected with an aqueous zoospore suspension of Alternaria solani in 2% biomalt solution having a density of 0.17×10⁶ spores/ml. The plants were then placed in a water-vapor-saturated chamber at temperatures of between 20 and 22° C. After 5 days, the infection on the leaves of the untreated, but infected control plants had developed to such an extent that the infection could be determined visually in %.

In this test, the plants which had been treated with 250 ppm of the title compound from example 1 were less than 5% infected, whereas the untreated plants were 80% infected.

### Use Example 2

### Activity Against Gray Mold on Bell Pepper Leaves Caused by Botrytis Cinerea

Bell pepper seedlings of the cultivar “Neusiedler Ideal Elite” were, after 2-3 leaves were well developed, sprayed to runoff point with an aqueous suspension having the concentration of active compound stated below. The next day, the treated plants were inoculated with a spore suspension of Botrytis cinerea which contained 1.7×10⁶ spores/ml in a 2% strength aqueous biomalt solution. The test plants were then placed in a climatized chamber at 22-24° C. and high atmospheric humidity. After 5 days, the extent of the fungal infection on the leaves could be determined visually in %.

In this test, the plants which had been treated with 250 ppm of the title compound from example 1 showed no infection, whereas the untreated plants were 90% infected.

1. A triazolopyrimidine of the formula I 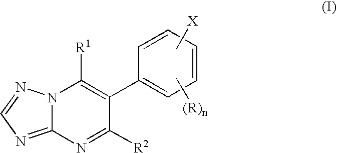
in which the index n and the substituents R, R¹, R² and X are as defined below:
X is nitro, a group —C(S)NR³R⁴, a group —C(═N—OR⁵)(NR⁶R⁷) or a group —C(═N—NR⁸R⁹)(NR¹⁰R¹¹),
R is halogen, cyano, hydroxyl, cyanato, C₁-C₈-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₈-cycloalkenyl, C₃-C₆-cycloalkoxy, C₁-C₈-alkoxycarbonyl, C₂-C₁₀-alkenyloxycarbonyl, C₂-C10-alkynyloxycarbonyl, aminocarbonyl, C₁-C₈-alkylaminocarbonyl, di(C₁-C₈-)alkylaminocarbonyl, C₁-C₈-alkoximinoalkyl, C₂-C₁₀-alkenyloximinocarbonyl, C₂-C₁₀-alkynyloximinoalkyl, C₁-C₈-alkylcarbonyl, C₂-C₁₀-alkenylcarbonyl, C₂-C₁₀-alkynylcarbonyl, C₃-C₆-cycloalkylcarbonyl, or a five- to ten-membered saturated, partially unsaturated or aromatic heterocycle which contains one, two, three or four heteroatoms from the group consisting of O, N and S;
R¹ is C₁-C₁₀-alkyl in which one carbon atom may be replaced by a silicon atom, C₂-C₁₀-alkenyl, C₂-C₁₀-alkynyl, C₃-C₁₂-cycloalkyl, C₃-C₁₀-cycloalkenyl, where the two lastmentioned groups may carry a C₁-C₄-alkylidene group, phenyl, naphthyl, or a five- to ten-membered saturated, partially unsaturated or aromatic heterocycle which is attached via a carbon atom and which contains one, two, three or four heteroatoms, independently of one another selected from the group consisting of O, N and S, as ring members;
where R¹ may be partially or fully halogenated or may carry one, two, three or four groups R^{a}, independently of one another selected from:
R^{a} halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₁-C₈-alkoximino, C₂-C₁₀-alkenyloximino, C₂-C₁₀-alkynyloximino, aryl-C₁-C₈-alkyloximino, C₂-C₁₀-alkynyl, C₂-C₁₀-alkenyloxycarbonyl, C₂-C₁₀-alkynyloxycarbonyl, phenyl, naphthyl, a five- to ten-membered saturated, partially unsaturated or aromatic heterocycle which contains one, two, three or four heteroatoms, independently of one another selected from the group consisting of O, N and S, as ring members,
where these aliphatic, alicyclic or aromatic groups for their part may be partially or filly halogenated or may carry one, two or three groups R^{b} independently of one another selected from:
R^{b} halogen, cyano, nitro, hydroxyl, mercapto, amino, carboxyl, aminocarbonyl, aminothiocarbonyl, alkyl, haloalkyl, alkenyl, alkenyloxy, alkynyl, alkynyloxy, alkoxy, haloalkoxy, alkylthio, alkylamino, dialkylamino, formyl, alkylcarbonyl, alkylsulfinyl, alkylsulfonyl, alkoxycarbonyl, alkylcarbonyloxy, alkylaminocarbonyl, dialkylaminocarbonyl, alkylaminothiocarbonyl and dialkylaminothiocarbonyl, where the alkyl groups in these radicals contain 1 to 6 carbon atoms and the alkenyl or alkynyl groups mentioned in these radicals contain 2 to 8 carbon atoms and the abovementioned groups may be partially or fully halogenated;
and/or 1, 2 or 3 of the following radicals:
cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C_{1-l -C}₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or may carry 1, 2, 3 or 4 alkyl or haloalkyl groups;
R² is C₁-C₄-alkyl, C₂-C₄-alkenyl or C₂-C₄-alkynyl, where the three lastmentioned groups may be unsubstituted or may carry 1, 2, 3, 4, 5 or 6 substituents independently of another selected from the group consisting of halogen, cyano, nitro, C₁-C₂-alkoxy and C₁-C₄-alkoxycarbonyl;
R³, R⁴, R⁵, R⁶, R⁷, R⁸, R⁹, R¹⁰ and R¹¹ are independently of one another selected from the group consisting of hydrogen, C₁-C₆-alkyl, C₃-C₆-cycloalkyl, C₂-C₆-alkenyl or C₂-C₆-alkynyl, where the 4 lastmentioned radicals may carry one, two, three, four, five or six radicals R^{a}; or
R³ and R⁴, R⁶ and R⁷, R⁸ and R⁹, and/or R¹⁰ and R¹¹ together with the nitrogen atom to which are attached form a four-, five- or six-membered saturated or partially unsaturated ring which may carry one, two, three or four substituents independently of one another selected from R^{a};
n is 0 or an integer 1, 2, 3 or 4.

2. The compound of the formula I, except for 5-methyl-6-(2-chloro-4-nitrophenyl)-7-(2-methylbutyl)-1,2,4-triazolo[1,5a]pyrimidine.

3. The compound of the formula I according to claim 1 in which X is nitro or a group —C(S)NR³R⁴.

4. The compound of the formula I according to claim 1 in which X is attached in the four-position relative to the point of attachment to the triazolopyrimidine skeleton.

5. The compound of the formula I according to claim 1 in which R is halogen, cyano, C₁-C₆-alkyl, C₁-C₆-haloalkyl, C₁-C₆-alkoxy, C₁-C₈-alkoxycarbonyl or aminocarbonyl.

6. The compound of the formula I according to claim 5 in which R is fluorine, chlorine, C₁-C₂-alkyl, C₁-C₂-alkoxycarbonyl, aminocarbonyl or C₁-C₂-fluoroalkyl.

7. The compound of the formula I according to claim 1 in which n is 0, 1 or 2.

8. The compound of the formula I according to claim 1 in which one of the radicals (R)ₙ is located in the 2-position relative to the point of attachment to the triazolopyrimidine skeleton.

9. The compound of the formula I according to claim 1 in which
R¹ is C₁-C₁₀-alkyl in which one carbon atom may be replaced by a silicon atom, C₃-C₈-alkenyl, C₃-C₈-alkynyl, C₃-C₆-cycloalkyl, C₅-C₆-cycloalkenyl, where the two lastmentioned groups may carry a C₁-C₄-alkylidene group, or is a 5- or 6-membered saturated or aromatic heterocycle which is attached via carbon; where R¹ may be partially or fully halogenated or may carry one, two, three or four identical or different groups R^{a}, in which
R^{a} is halogen, cyano, C₁-C₆-alkyl, C₂-C₆-alkenyl, C₂-C₆-alkynyl, C₁-C₆-alkoxy, C₁-C₆-alkoxycarbonyl, C₁-C₆-alkoximino, C₂-C₆-alkenyloximino, C₂-C₆-alkynyloximino, C₃-C₆-cycloalkyl, C₅-C₆-cycloalkenyl, where the aliphatic or alicyclic groups for their part may be partially or fully halogenated or may carry one, two or three groups R^{b}:
R^{b} is halogen, cyano, C₁-C₆-alkyl, C₁-C₆-haloalkyl, C₂-C₆-alkenyl, C₂-C₆-alkynyl, C₁-C₆-alkylcarbonyl, C₁-C₆-haloalkylcarbonyl or C₁-C₆-alkoxy.

10. The compound of the formula I according to claim 9 in which
R¹ is C₁-C₈-alkyl, C₁-C₆-haloalkyl, C₃-C₈-alkenyl, C₃-C₆-haloalkenyl, C₃-C₈-alkynyl, C₁-C₄-alkyl-C₃-C₆-cycloalkyl, C₃-C₆-cycloalkyl or C₁-C₄-alkyl-C₅-C₆-cycloalkenyl.

11. The compound of the formula I according to claim 1 in which
R² is C₁-C₄-alkyl which may be substituted by halogen, cyano, nitro or C₁-C₂-alkoxy.

12. The compound of the formula I according to claim 11 in which R² is C₁-C₄-alkyl or C₁-C₄-haloalkyl.

13. The use of a compound of the formula I according to claim 1 for controlling phytopathogenic fungi.

14. A composition for controlling phytopathogenic fungi, which composition comprises at least one compound of the formula I according to claim 1 and/or an agriculturally acceptable salt of I and at least one solid or liquid carrier.

15. A method for controlling phytopathogenic fungi, wherein the fungi or the materials, plants, the soil or seed to be protected against fungal attack are treated with an effective amount of a compound of the formula I according to claim 1 and/or an agriculturally acceptable salt of I.

16. The compound of the formula I according to claim 2 in which X is attached in the four-position relative to the point of attachment to the triazolopyrimidine skeleton.

17. The compound of the formula I according to claim 3 in which X is attached in the four-position relative to the point of attachment to the triazolopyrimidine skeleton.

18. The compound of the formula I according to claim 2 in which R is halogen, cyano, C₁-C₆-alkyl, C₁-C₆-haloalkyl, C₁-C₆-alkoxy, C₁-C₈-alkoxycarbonyl or aminocarbonyl.

19. The compound of the formula I according to claim 3 in which R is halogen, cyano, C₁-C₆-alkyl, C₁-C₆-haloalkyl, C₁-C₆-alkoxy, C₁-C₈-alkoxycarbonyl or aminocarbonyl.

20. The compound of the formula I according to claim 4 in which R is halogen, cyano, C₁-C₆-alkyl, C₁-C₆-haloalkyl, C₁-C₆-alkoxy, C₁-C₈-alkoxycarbonyl or aminocarbonyl.
